(19) United States
(12) Patent Application Publication
(10) Pub. No.: US 2026/0001922 A1
(43) Pub. Date: Jan. 1, 2026

(54) PEPTIDES TARGETING SODIUM CHANNELS TO TREAT PAIN

(71) Applicant: The Regents of the University of California, Oakland, CA (US)

(72) Inventors: Vladimir YAROV-YAROVOY, Oakland, CA (US); Heike WULFF, Oakland, CA (US); Phuong T. NGUYEN, Oakland, CA (US); Hai M. NGUYEN, Oakland, CA (US); Karen WAGNER, Oakland, CA (US); Jon T. SACK, Oakland, CA (US); Bruce HAMMOCK, Oakland, CA (US)

(21) Appl. No.: 18/881,483

(22) PCT Filed: Jul. 5, 2023
(86) PCT No.: PCT/US23/69630

§ 371 Date: Jan. 6, 2025

Related U.S. Application Data
(60) Provisional application No. 63/358,684, filed on Jul. 6, 2022.

(51) Int. Cl. C07K 14/435; A61K 38/00; A61P 25/04

(52) U.S. Cl. CPC C07K 14/43518; A61P 25/04; A61K 38/00

(57) ABSTRACT

The present invention describes human Na_{V}1.7 inhibitor peptides, compositions, and methods for using the peptides for treating pain.

### CROSS-REFERENCES TO RELATED APPLICATIONS

This application claims priority to U.S. Provisional Application No. 63/358,684, filed Jul. 6, 2022, which is incorporated herein in its entirety for all purposes.

### STATEMENT OF GOVERNMENTAL SUPPORT

This invention was made with government support under Grant No. UG3 NS114956 awarded by the National Institute of Neurological Disorders and Stroke of the National Institutes of Health of the United States of America. The government has certain rights in the invention.

### BACKGROUND OF THE INVENTION

More than 25 million Americans suffer from chronic pain. Chronic pain originates from tissue or nervous system damage and persists longer than three months. The many causes of chronic pain include surgery, chemotherapy, complex regional pain syndrome, and back pain. People with chronic pain experience higher anxiety, depression, sleep disturbances, and gain weight due to decreased physical activity. Non-opioid treatment options for chronic pain are limited. Inhibitors of neuronal ion channels are important alternatives that have not demonstrated addiction liability. Non-selective Nav channel inhibitors, including carbamazepine, lacosamide, and lamotrigine are used among initial options to treat patients with chronic pain. For example, intravenous infusion of the local anesthetic lidocaine, a non-specific Nav channel inhibitor, reduces chronic pain in some patients. However, lidocaine treatments have serious side effects including cardiac arrest, abnormal heartbeat, and seizures. Patients with chronic pain who are not responding to Nav channel inhibitors can be prescribed opioids, but the severe side effects of opioids such as constipation, respiratory depression, and addiction limit their utility. Intrathecal infusion of the voltage-gated calcium channel inhibitor ziconotide is also effective against chronic pain. However, ziconotide has serious psychiatric side effects. Consequently, the treatment of chronic pain remains a major unmet medical need. Nav channels have been thoroughly clinically validated as pharmacological targets for pain treatment, but currently available therapies are limited by incomplete efficacy and significant side effects.

Nociceptive signals originate in peripheral nerve fibers that transduce chemical, mechanical, or thermal stimuli into action potentials that propagate along their axons to the synaptic nerve terminals in the spinal dorsal horn. Voltage-gated sodium (Nav) channels are key molecular determinants of action potential generation and propagation in excitable cells. Of the nine known human Nav (hNav) channel subtypes, genetic and functional studies identified three subtypes as important for pain signaling: Nav1.7, Nav1.8, and Nav1.9, which are predominantly expressed in peripheral neurons. Nav1.7 possesses a slow closed-state inactivation compared to other channels, making it uniquely important for setting the threshold for action potential firing, and thus the gain in pain signaling neurons. In accordance with this, loss-of-function mutations in hNav1.7 have been identified in families with congenital insensitivity to pain. Gain-of-function mutations in hNav1.7 lead to inherited pain disorders; families with inherited erythromelalgia have hNav1.7 mutations that shift its voltage-dependence of activation to hyperpolarized voltages, leading to hyperexcitability in dorsal root ganglion (DRG) neurons and chronic neuropathic pain; patients with paroxysmal extreme pain disorder have defects in hNav1.7 fast inactivation resulting in persistent sodium currents and episodic burning pain. These and other studies have validated hNav1.7 as a prime target for the treatment of pain. Disruptions in normal Nav channel function can have dramatic effects on physiological neuronal signaling and lead to failure of electrical signaling, increased sensitivity to pain, congenital indifference to pain, uncoordinated movement, seizures, or paralysis.

Mammalian Nav channels are composed of four homologous domains (I through IV), each containing six transmembrane segments (S1 through S6), with segments S1-S4 of the channel forming the voltage-sensing domain (VSD) and segments S5 and S6 forming the pore. The binding of local anesthetics to a receptor site formed within the pore inner cavity can directly block ion conduction through the Nav channels. However, because of the high conservation of residues forming this local anesthetic receptor site among the different isoforms, all currently available therapeutic drugs targeting Nav channels are non-specific.

More than ten years ago scientists at Merck demonstrated that a peptide from the venom of the Chilean tarantula Thrixopelma pruriens, termed Protoxin-II (ProTx-II), selectively targeted the Nav1.7 channel subtype and blocked action potential propagation in nociceptors. Amgen also developed peptide inhibitors of Nav1.7 and identified a novel peptide toxin from the venom of the Chilean tarantula Grammostola porteria, termed GpTx-1, which was a less potent inhibitor of human Nav1.7, compared to ProTx-II, but had 20-fold and 1,000 fold selectivity against Nav1.4 (predominantly expressed in muscle) and Nav1.5 (predominantly expressed in the heart). Using the GpTx-1 NMR structure as a guide, Amgen scientists created a variant with improved potency and selectivity than the wild-type toxin, concluding that GpTx-1 variants can potentially be further developed as peptide therapeutics. Janssen Biotech demonstrated that ProTx-II exerted a strong analgesic effect following intrathecal injection in rat models of thermal and chemical nociception. While efficacious, ProTx-II had a narrow therapeutic window, and induced profound motor effects at moderately higher doses, consistent with inhibition of Nav channel subtypes present on motor neurons (Nav1.1 and Nav1.6). Janssen Biotech pursued resource-intensive optimization of ProTx-II, but without a structure to guide optimization. This blind optimization process produced 1,500 ProTx-II variants, including a peptide, named JNJ63955918, with improved 100-fold selectivity for Nav1.7 over all other Nav channel subtypes tested. However, JNJ63955918 had ˜10-fold reduced affinity for Nav1.7. The in vivo safety window for JNJ63955918 was 7-16-fold, limited by motor deficits and muscle weakness, consistent with insufficient selectivity against off-target Nav channels.

Other variations of GpTx-1 or ProTx-II have been reported. See, U.S. Pat. Nos. 9,624,280, 9,279,003, 9,636,418, and 10344060, and US patent application publication nos. 20160222071, 20180105561, and 20180022786, each of which is incorporated by reference in its entirety.

There continues to be a need to develop potent and selective Nav inhibitors that can be used to treat pain. This invention addresses this and other needs.

### BRIEF SUMMARY OF THE INVENTION

In some embodiments, a peptide of the present invention is a peptide comprising Formula I:

(I)
(SEQ ID NO: 101)
X¹-X²-X³-K⁴-X⁵-X⁶-X⁷-X⁸-X⁹-D¹⁰-X¹¹-X¹²-R¹³-K¹⁴-
X¹⁵-X¹⁶-X¹⁷-G¹⁸-X¹⁹-R²⁰-X²¹-X²²-L²³-W²⁴-X²⁵-X²⁶-
X²⁷-X²⁸-X²⁹-X³⁰,

or a pharmaceutically acceptable salt thereof,
wherein
X¹ is Q, H, R, K, P, or Y;
X² is C or Sec;
X³ is Q or L;
X⁵ is W or A;
X⁶ is M, Nle, or F;
X⁷ is Q or W;
X⁸ is T orQ;
X⁹ is C or Sec;
X¹¹ is K, R, or S;
X¹² is D, A, T, S, or E;
X¹⁵ is C or Sec;
X¹⁶ is C or Sec;
X¹⁷ is E, D, A, or P;
X¹⁹ is F, norleucine (Nle), or L;
X²¹ is C or Sec;
X²² is R or norarginine (NorR);
X²⁵ is C or Sec;
X²⁶ is R or K;
X²⁷ is K or 2,4-dimethylphenylalanine (diMePhe);
X²⁸ is E or Q;
X²⁹ is L or tert-butylcysteine (tBuCys);
X³⁰ is L, A, C, D, E, F, G, H, I, K, M, N, P, Q, R, S, T, V, W, or Y, or is absent;
when X² and X¹⁶ are each C, the —SH groups between X² and X¹⁶ are combined to form a disulfide bond, or alternatively, X² and X¹⁶ each comprise a —SH group;
when X⁹ and X²¹ are each C, the —SH groups between X⁹ and X²¹ are combined to form a disulfide bond, or alternatively, X⁹ and X²¹ each comprise a —SH group;
when X¹⁵ and X²⁵ are each C, the —SH groups between X¹⁵ and X²⁵ are combined to form a disulfide bond, or alternatively, X¹⁵ and X²⁵ each comprise a —SH group; and
the C-terminus has a —C(O)NH₂, or alternatively, the C-terminus has a —C(O)OH.

In some embodiments, the pharmaceutical composition of the present invention is a pharmaceutical composition comprising a peptide as described herein, or pharmaceutically acceptable salt thereof, and a pharmaceutically acceptable excipient.

In some embodiments, the method of the present invention is a method of treating pain in a subject in need thereof, comprising administering to the subject a therapeutically effective amount of a peptide as described herein, or a pharmaceutically acceptable salt thereof.

### BRIEF DESCRIPTION OF THE DRAWINGS

FIG. 1A-1B show a computational analysis of the residues of human Nav1.7 in contact with ProTx-II (SEQ ID NO: 20). FIG. 1A shows a Rosetta visualization of ProTx-II in the binding site of human Nav1.7. FIG. 1B shows contacts between each amino acid residue of ProTx-II (y-axis from residue 1 to 30) and either the lipid head, lipid tail, VSD-II, or exposed (“water”).

FIG. 2A-2B show computational design of new peptides based on the Rosetta analysis of ProTx-II (SEQ ID NO: 20) binding to human Nav1.7. FIG. 2A depicts consensus designed sequences based on literature ProTx-II variants. FIG. 2B shows the top 20 peptides design1 (SEQ ID NO: 21) through design20 (SEQ ID NO: 40) designed using Rosetta for testing and evaluation.

FIG. 3 shows a sequence alignment of ProTx-II (SEQ ID NO: 20) with other literature peptides.

FIG. 4A-4D show the results of the first optimization round. FIG. 4A: Sequence alignment of the wild-type ProTx-II (SEQ ID NO: 20) with PTx2-2954 (SEQ ID NO: 14) and PTx2-2955 (SEQ ID NO: 8) peptides. FIG. 4B: Transmembrane (left panel) and extracellular (right panel) views of the PTx2-2955 (SEQ ID NO: 8)—hNav1.7 model. Key residues on the PTx2-2955 (SEQ ID NO: 8) and hNav1.7 are shown in stick representation and labeled. Nitrogen atom are colored in blue and oxygen atoms are colored in red. Hydrogen bonds between donor and acceptor atoms are shown by blue dash line. FIG. 4C: Block of whole-cell hNav1.7 sodium currents by application of increasing concentrations of PTx2-2955 (SEQ ID NO: 8) and followed by 1 mM of wild-type ProTx-II as indicated. FIG. 4D: Inhibition of hNav1.7 currents was measured as shown in FIG. 4C and plotted as a function of WT ProTx-2 or PTx2-2955 (SEQ ID NO: 8) concentration. Fitting the Hill equation to the data yielded IC₅₀ values (95% confidence interval) of 1.7 [0.5, 2.9] nM (n=3) for WT ProTx-II and 185.0 [152.1, 217.9] nM (n=5) for PTx2-2955, respectively.

FIG. 5 shows a sequence alignment of the binding regions of human Nav1.1 through human Nav1.9.

FIG. 6A-6D show the results of the 2^{nd} optimization round. FIG. 6A: Sequence alignment of the wild-type ProTx-II (SEQ ID NO: 20) with PTx2-2955 (SEQ ID NO: 8) and PTx2-3063-PTx2-3067 peptides. FIG. 6B: Transmembrane (left panel) and extracellular (right panel) views of the PTx2-3066 (SEQ ID NO: 12)—hNav1.7 model. Key residues on the PTx2-3066 (SEQ ID NO: 12) and hNav1.7 are shown in stick representation and labeled. Nitrogen atom are colored in blue and oxygen atoms are colored in red. Hydrogen bonds between donor and acceptor atoms are shown by blue dash line. FIG. 6C: Block of whole-cell hNav1.7 sodium currents by application of increasing concentrations of PTx2-3066 (SEQ ID NO: 12). FIG. 6D: Inhibition of hNav1.7 currents was measured as shown in FIG. 6C and plotted as a function concentration of PTx2-2955 (SEQ ID NO: 8) or its derivatives. Fitting the Hill equation to the data yielded IC₅₀ values (95% confidence interval) of 185.0 [152.1, 217.9] nM (n=5), 154.0 [39.9, 268.1] nM (n=3), nM, 52.6 [7.0, 98.2] nM (n=3), 73.9 [55.8, 92.0] nM (n=4), 30.8 [27.9, 33.7] nM (n=6), and 48.3 [29.5, 67.1] nM (n=4) for PTx2-2955 (SEQ ID NO: 8), PTx2-3063 (SEQ ID NO: 9), PTx2-3064 (SEQ ID NO: 10), PTx2-3065 (SEQ ID NO: 11), PTx2-3066 (SEQ ID NO: 12), and PTx2-3067 (SEQ ID NO: 13), respectively.

FIG. 7 shows percent inhibition (“% inhibition”) of PTx-3064 (SEQ ID NO: 10) and PTx-3066 (SEQ ID NO: 12) peptides at 10 μM on human Nav1.2, human Nav1.4, or human Nav1.5. At 10 μM, PTx2-3064 (SEQ ID NO: 10) blocked ˜92%, ˜66%, and ˜25% of currents conducted by hNav1.2, hNav1.4, and hNav1.5, respectively. At the same concentration, PTx2-3066 (SEQ ID NO: 12) blocked ˜41%, ˜34%, and ˜1% of currents conducted by hNav1.2, hNav1.4, and hNav1.5, respectively. Data are represented as mean standard deviation derived from 3 individually recorded cells.

FIG. 8A-8D show the results of the 3^{rd} optimization round. FIG. 8A: Sequence alignment of the wild-type ProTx-II (SEQ ID NO: 20) with PTx2-3066 (SEQ ID NO: 12), PTx2-3127 (SEQ ID NO: 1), and PTx2-3128 (SEQ ID NO: 7) peptides. FIG. 8B: Transmembrane (left panel) and extracellular (right panel) views of the PTx2-3127-hNav1.7 model. Key residues on the PTx2-3127 (SEQ ID NO: 1) and hNav1.7 are shown in stick representation and labeled. Nitrogen atom are colored in blue and oxygen atoms are colored in red. Hydrogen bonds between donor and acceptor atoms are shown by blue dash line. FIG. 8C: Block of whole-cell hNav1.7 sodium currents by application of increasing concentrations of PTx2-3127. FIG. 8D: Inhibition of hNav1.7 currents was measured as shown in FIG. 8C and plotted as a function concentration of PTx2-3066 or its derivatives. Fitting the Hill equation to the data yielded the IC₅₀ values (95% confidence interval) of 30.8 [27.9, 33.7] nM (n=6), 2.3 [1.9, 2.7]μM (n=3), 6.9 [6.7, 7.1] nM (n=3), and 5.0 [4.6, 5.4] nM (n=3) for PTx2-3066, PTx2-3126, PTx2-3127, PTx2-3128, respectively.

FIG. 9A-9D show the results of the 4^{th} optimization round. FIG. 9A: Sequence alignment of the wild-type ProTx-II (SEQ ID NO: 20) with PTx2-3127 (SEQ ID NO: 1), PTx2-3258 (SEQ ID NO: 2), PTx2-3259 (SEQ ID NO: 4), PTx2-3260 (SEQ ID NO: 5), and PTx2-3361 (SEQ ID NO: 3) peptides. FIG. 9B: Transmembrane (left panel) and extracellular (right panel) views of the PTx2-3258 (SEQ ID NO: 2)—hNav1.7 model. Key residues on the PTx2-3258 (SEQ ID NO: 2) and hNav1.7 are shown in stick representation and labeled. Nitrogen atom are colored in blue and oxygen atoms are colored in red. Hydrogen bonds between donor and acceptor atoms are shown by blue dash line. FIG. 9C: Block of whole-cell hNav1.7 sodium currents by application of increasing concentrations of PTx2-3258 (SEQ ID NO: 2) and followed by 1 mM of wild-type ProTx-II as indicated. FIG. 9D: Inhibition of hNav1.7 currents was measured as shown in FIG. 9C and plotted as a function concentration of PTx2-3127 (SEQ ID NO: 1) or its derivatives. Fitting the Hill equation to the data yielded the IC₅₀ values of 7 nM, 4 nM, 40 nM, 21 nM, 9 nM for PTx2-3127 (SEQ ID NO: 1), PTx2-3258 (SEQ ID NO: 2), PTx2-3259 (SEQ ID NO: 4), PTx2-3260 (SEQ ID NO: 5), and PTx2-3361 (SEQ ID NO: 3), respectively.

FIG. 10A-10G show the efficacy of designed Nav1.7-selective inhibitor (PTx2-3127) (SEQ ID NO: 1) on Nav channels of mouse nonpeptidergic nociceptor neurons. FIG. 10A: Immunofluorescence from MrgprD^{GFP} labeled NP1 nociceptors (AB_300798, green) and Nay1.7 (AB_2877500, magenta) in a mouse L5 spinal section. Orientation of left DRG was moved during sectioning. Lower panels are zoomed in images to highlight colocalization (white) in dorsal horn nociceptor terminals, dorsal root fibers and DRG cell bodies. NP1 nociceptor DRG cell bodies show both high (arrow) and low (arrowhead) immunofluorescence for Nav1.7. Top image, dorsal horn and DRG zoom images are a z-projection of 3 confocal images spanning 10.06 μm. Zoom in image of dorsal root fibers is a z-projection of 9 airyscan images spanning 3.18 μm. Scale bar in the top image is 500 μm. Scale bars in the dorsal horn, dorsal root and DRG zoom in panels are 100, 20 and 100 μm respectively. FIG. 10B: Voltage clamp recordings of Nav currents from dissociated NP1 nociceptors showing impact of PTx2-3127 (red) and subsequent application of TTX (green). Fast-inactivating Nav component revealed by subtraction of 1 μM PTx2-3127 trace from total Nav current. Black dotted line represents 0 pA of current. FIG. 10C: Left: Mean current density from 0.4-1 ms of PTx2-3127 sensitive current and vehicle sensitive current. Middle: Mean current density from 0.4-1 ms of TTX sensitive current after application of PTx2-3127 or vehicle. Right: Peak current density of TTX resistant current after application of PTx2-3127 or vehicle and TTX. Point colors represent the same neuron (N=4 mice). p values calculated by Students T-Test. FIG. 10D: Peak time of PTx2-3127 sensitive and resistant currents as well as peak time of TTX sensitive and resistant currents. Point colors correspond to the same neurons and is consistent with points shown in FIG. 10C. p values calculated by Students T-Test. FIG. 10E: Current clamp recording ofNP1 action potentials and failures with 3 Hz stimuli in vehicle, 1 μM PTx2-3127 and 1 μM TTX. FIG. 10F: Average remaining NP1 action potentials (APs) versus frequency in PTx2-3127 (red points, n=8 neurons, N=4 mice) or in vehicle control (blue points, n=8 neurons, N=4 mice). Average remaining APs after PTx2-3127 or vehicle control in 1 μM TTX (red circle green fill and blue circle green fill respectively). Neurons with no sensitivity to TTX were excluded from this analysis. FIG. 10G: Rheobase of NP1 neurons before PTx2-3127, in PTx2-3127 and in TTX (left). Rheobase of NP1 neurons before vehicle, in vehicle and in TTX (right). p values calculated by Students T-test.

FIG. 11A-11B show results of peptides of the invention in mouse DRG neurons. FIG. 11A: Current clamp recording of TTX insensitive NP1 action potentials with 3 Hz stimuli in vehicle, 1 μM PTx2-3127 and 1 μM TTX. FIG. 11B: Rheobase of TTX insensitive NP1 neurons before PTx2-3127 or vehicle and in TTX. Red points and lines indicate that neurons were in PTx2-3127 before TTX while blue points and lines indicate that neurons were in vehicle before TTX. p values calculated by Students T-test.

FIG. 12 shows efficacy of PTx2-3127 (SEQ ID NO: 1) on rheobase and action potentials in human DRG neurons. Rheobase and action potential inhibition by PTx2-3127 following 24 h incubation with Oxaliplatin (50 μM). Rheobase (top) and action potential inhibition (bottom) after perfusion of compound are normalized to baseline. APs were elicited at 150% of baseline rheobase. Results are presented as mean SEM.

FIG. 13A-13C show stability of peptides in artificial cerebrospinal fluid (aCSF). FIG. 13A: wild type ProTx-II (SEQ ID NO: 20); FIG. 13B: PTx-3127 (SEQ ID NO: 1); FIG. 13C: PTx-3258 (SEQ ID NO: 2).

FIG. 14A-14C show efficacy of PTx2-3127 (SEQ ID NO: 1) on thermal pain and CIPN neuropathy. PTx2-3127 (SEQ ID NO: 1) exhibited dose dependent analgesia on a 52.1° C. hotplate increasing the duration of effect as well as number reaching the latency cutoff with doses of 1.2 ug i.t. (FIG. 14A) to 1.6 ug i.t. (FIG. 14B) in naïve rats. The analgesia mediated by PTx2-3127 (SEQ ID NO: 1) was significant compared to vehicle controls for both doses (1.2 ug, p≤0.002) and the 1.6 ug dose several rats reached the hotplate latency cutoff (30 seconds to prevent injury) for several hours' duration (1.6 ug, p≤0.001 and p=0.013 at indicated time points). FIG. 14C: PTx2-3127 (SEQ ID NO: 1) was also effective against oxaliplatin chemotherapy induced neuropathic pain (CIPN) with responses also significant compared to vehicle controls (p≤0.001) and reaching the latency cutoff. (FIGS. 14A-14C, Two Way Repeated Measures ANOVA, Holm-Sidak method post hoc, treated versus control).

FIG. 15A-15C show exemplary activity of PTx-3127 (SEQ ID NO: 1). FIG. 15A: Plots of current-voltage relationship of normalized hNav1.7 currents measured in control cells (black, n=10) and cells exposed to 50 nM of PTx2-3127 (red, n=8). Cells were stepped in 5-mV increments from −120 mV to +70 mV from a holding potential of −120 mV for 10 ms. FIG. 15B: Voltage-dependent activation curves are derived from the data shown in FIG. 15A. PTx2-3127 causes a statistically significant depolarized shift in steady-state activation in the depolarizing direction. For control cells, the V1/2 of activation is −28.1±0.9 mV, and the slope factor k is 5.0±0.5 mV; for PTx2-3127-treated cells, the V1/2 of activation is −17.3±3.9 mV, and the slope factor k is 2.4±0.2 mV. FIG. 15C: Normalized steady-state inactivation curves measured control cells (black, n=12) and cells exposed to 50 nM PTx2-3127 (red, n=6). PTx2-3127 causes a statically significant shift in steady-state inactivation in the hyperpolarized direction. For control cells, the V1/2 of inactivation is −71.2±0.9 mV, and the slope factor k is 5.8±0.1 mV; for PTx2-3127-treated cells, the V1/2 of inactivation is −76.2±2.5 mV, and the slope factor k is 7.3±0.3 mV. Cells were stepped in 10-mV increments from −120 mV to 30 mV for 500 ms followed by a test pulse to −10 mV for 30 ms. All recordings were performed in a time-matched manner, and normalized conductances and currents were fit to a Boltzmann function, and are shown as means t SEM.

### DETAILED DESCRIPTION OF THE INVENTION

### I. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“About” when referring to a value includes the stated value+/−10% of the stated value. For example, about 50% includes a range of from 45% to 55%, while about 20 molar equivalents includes a range of from 18 to 22 molar equivalents. Accordingly, when referring to a range, “about” refers to each of the stated values+/−10% of the stated value of each end of the range. For instance, a ratio of from about 1 to about 3 (weight/weight) includes a range of from 0.9 to 3.3.

An “amino acid” used in the invention includes one that is available commercially or available by routine synthetic methods. Certain amino acids that may require special methods for incorporation into the peptide, and sequential, divergent or convergent synthetic approaches to the peptide sequence are useful in this invention. An amino acid can be a D-amino acid or an L-amino acid. The present disclosure is meant to include all such possible isomers, as well as their racemic and optically pure forms. When not naturally occurring, optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Where peptides are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such peptide(s). As used herein, “scalemic mixture” is a mixture of stereoisomers at a ratio other than 1:1. When there is no indication, the L isomer is used.

Natural coded amino acids are represented by either the one-letter code or three-letter codes according to IUPAC conventions and as found in Table 1 below.

TABLE 1
Natural Amino Acids.

| Amino Acid | One-Letter Code | Three-Letter Code |
|---|---|---|
| L-alanine | A | Ala |
| L-cysteine | C | Cys |
| L-aspartic acid | D | Asp |
| L-glutamic acid | E | Glu |
| L-phenylalanine | F | Phe |
| glycine | G | Gly |
| L-histidine | H | His |
| L-isoleucine | I | Ile |
| L-lysine | K | Lys |
| L-leucine | L | Leu |
| L-methionine | M | Met |
| L-asparagine | N | Asn |
| L-proline | P | Pro |
| L-glutamine | Q | Gln |
| L-arginine | R | Arg |
| L-serine | S | Ser |
| L-threonine | T | Thr |
| L-selenocysteine | U | Sec |
| L-valine | V | Val |
| L-tryptophan | W | Trp |
| L-tyrosine | Y | Tyr |

When D-amino acids are used, the lower case one or three letter codes of the amino acids are designated. For example, D-valine can be abbreviated as v or val. Hence, three letter codes of D-amino acids include ala, cys, asp, glu, phe, his, ile, lys, leu, met, asn, pro, gin, arg, ser, thr, sec, val, trp, and tyr. The corresponding one letter codes of D-amino acids include a, c, d, e, f, h, i, k, l, m, n, p, r, s, t, u, v, w, and y.

Cysteine (Cys) free thiol and disulfide forms are included in the peptides of the invention. As understood in the art, an L-cysteine (Cys) amino acid in a peptide can exist in free thiol form, that is, comprising a —SH group and having the structure:

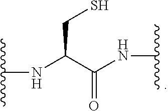 [SMILES: *NC(=O)[C@H](CS)N*]

Alternatively, Cys can form a disulfide bond with another Cys. The disulfide bond can be intramolecular. Hence, a peptide with two Cys in which the —SH groups combine to form a disulfide bond can have the structure:

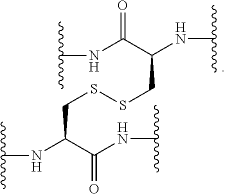 [SMILES: *NC(=O)[C@H](CSSC[C@H](N*)C(=O)N*)N*]

Non-natural amino acids are known in the art and can be included in the peptides of the invention. Exemplary non-natural amino acids include the following:

| Amino Acid | Structure | Abbreviation |
|---|---|---|
| L-2,4- dimethylphenylalanine | 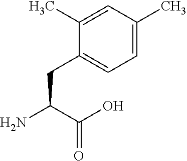 | diMePhe |
| L-norarginine | 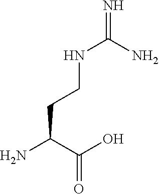 [SMILES: N=C(N)NCC[C@H](N)C(=O)O] | NorR |
| L-norleucine | 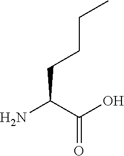 | Nle |
| L-tert-butylcystcinc | 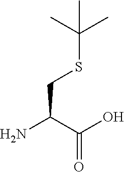 | tBuCys |

“Peptide,” “polypeptide,” and “protein” are used interchangeably herein, and refer to naturally occurring and synthetic amino acids of any length, as well as amino acid analogs and amino acid mimetics that function in a manner similar to the naturally occurring amino acids. The term “peptide” includes fusion proteins, including, but not limited to, fusion proteins with a heterologous amino acid sequence, fusions with heterologous and homologous leader sequences, with or without N-terminal methionine residues; immunologically tagged proteins; and the like. Peptides further include post-translationally modified peptides. Amino acid sequences of peptides are recited from N- to C-terminus as is common in the art.

The peptides of the present invention may incorporate additional N- and/or C-terminal amino acids when compared to the peptide of Formula I (SEQ ID NO: 101), for example resulting from cloning and/or expression schemes.

In some embodiments, the peptides of the present invention or a pharmaceutically acceptable salt thereof is derivatized. In some embodiments, the peptide is derivatized at an N-terminal amino acid. Non-limiting examples of moieties with which the N-terminal (first) amino acid can be derivatized include an alkyl group (such as C₁-C₄ alkyl), a methyl group, a carboxy group, an acetyl group, and a substituted acetyl group. In some embodiments, the peptide is derivatized at a C-terminal amino acid. Non-limiting examples of chemical moieties with which the C-terminal (last) amino acid can be derivatized include an alkyl group (such as C₁-C₄ alkyl, e.g. a methyl or ethyl group); an aryl group or aryl alkyl group, such as phenyl or benzyl; a halogen, such as a fluoro or chloro; an alkoxy group; and an amino group.

The peptides of the present invention may incorporate one or more further modifications when compared to the peptide of the present invention, such as a peptide of Formula I (SEQ ID NO: 101), for example, by incorporating a fluorescent label. In some embodiments, fluorescently labeled peptides can be used for in vivo biomedical imaging, protein binding and localization studies. Fluorochrome-conjugated peptides may be visualized by fluorescence microscopy or other fluorescence visualization techniques. The fluorescent label can be covalently attached at the N-terminus, the C-terminus, or to an amino acid side-chain anywhere in the peptide. In some embodiments, the fluorescent label is a thiol-reactive fluorescent dye (for example, 5-(2-((iodoacetyl)amino)ethyl) aminonapthviene-1-sulfonic acid (1,5-IEDANS) or fluorescein) or is chosen from the light-emitting moieties, dipyrromethene boron fluoride (Bodipy), fluorescein thiosemicarbazide (FTC), sulforhodamine 101 acid chloride (Texas Red), phycoerythrin rhodamine, carboxytetramethylrhodamine, 4,6-diamino-2-phenylindole (DAPI), an indopyras dye, pyrenyloxytrisulfonic acid (Cascade Blue, 514 carboxylic (Oregon Green), eosin, erythrosin, pyridyloxazole, benzoxadiazole, aminonapthalene, pyrene, maleimide, a coumarin, 4-fluoro-7-nitrobenfurazan (NBD), 4-amino-N[3-(vinylsulfonyl)-(phenyl]napthalimide-3,6-disulfonate) (Lucifer Yellow, propidium iodide, a porphyrin, a cyanine dye (CY³, CY⁵, CY⁹, a lanthanide cryptate, a lanthanide chelate, or a derivative or analog thereof. In some embodiments, the fluorescent label is covalently bonded to a Cys —SH group.

In some embodiments, peptides are described herein or pharmaceutically acceptable salts, isomers, or a mixture thereof, in which from 1 to n hydrogen atoms attached to a carbon atom may be replaced by a deuterium atom, in which n is the number of hydrogen atoms in the molecule. As known in the art, the deuterium atom is a non-radioactive isotope of the hydrogen atom. Such peptides may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism”, Trends Pharmacol. Sci., 5(12):524-527 (1984). Such peptides are synthesized by means well known in the art, for example by employing starting materials in which one or more hydrogen atoms have been replaced by deuterium.

Examples of isotopes that can be incorporated into the disclosed peptides also include isotopes of hydrogen, carbon, nitrogen, oxygen, phosphorous, fluorine, chlorine, and iodine, such as ²H, ³H, ¹¹C, ¹³C, ¹⁴C, ¹³N ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled peptides of the present invention, such as a peptide of Formula I (SEQ ID NO: 101), can generally be prepared by conventional techniques known to those skilled in the art or by processes analogous to those described in the Examples as set out below using an appropriate isotopically-labeled reagent in place of the non-labeled reagent previously employed.

In some embodiments, the N-terminus of a peptide of the present invention or pharmaceutically acceptable salt thereof is unmodified.

In some embodiments, a C-terminus of a peptide of the present invention or pharmaceutically acceptable salt thereof is unmodified, thereby displaying a carboxylate (—C(O)OH). For such peptides of the present invention, the corresponding C-terminus has a —C(O)OH. For instance, an LL-containing peptide wherein the C-terminus has a —C(O)OH refers to the structure:

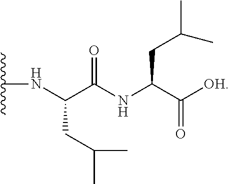 [SMILES: *N[C@@H](CC(C)C)C(=O)N[C@@H](CC(C)C)C(=O)O]

In some embodiments, the C-terminus of a peptide of the present invention or pharmaceutically acceptable salt thereof is modified, for example, by converting the carboxylate to a C-terminal primary amide (—C(O)NH₂). Such peptides of the invention display a C-terminus that has a —C(O)NH₂. For instance, an LL-containing peptide wherein the C-terminus has a —C(O)NH₂ refers to the structure:

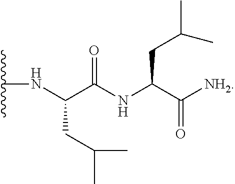 [SMILES: *N[C@@H](CC(C)C)C(=O)N[C@@H](CC(C)C)C(N)=O]

Provided are also pharmaceutically acceptable salts of the peptides described herein. “Pharmaceutically acceptable” or “physiologically acceptable” refer to peptides, salts, compositions, dosage forms and other materials which are useful in preparing a pharmaceutical composition that is suitable for veterinary or human pharmaceutical use.

“Pharmaceutical composition” as used herein refers to a product comprising the specified ingredients in the specified amounts, as well as any product, which results, directly or indirectly, from combination of the specified ingredients in the specified amounts. The pharmaceutical composition is generally safe for biological use.

“Pharmaceutically acceptable excipient” as used herein refers to a substance that aids the administration of an active agent to an absorption by a subject. Pharmaceutically acceptable excipients useful in the present invention include, but are not limited to, binders, fillers, disintegrants, lubricants, coatings, sweeteners, flavors and colors. One of skill in the art will recognize that other pharmaceutically acceptable excipients are useful in the present invention.

“Treatment” or “treat” or “treating” as used herein refers to an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

“Pain” refers to any type of pain in the art, including, for example, peripheral and central neuropathic pain, functional pain, inflammatory pain or nociceptive pain, whether acute or chronic.

“Subject” as used herein refers to a mammal, including veterinary mammals such as a mouse, rat, dog, or cat; livestock such as a lamb, goat, horse, donkey, or cow; and primates such as monkeys, for example, cynomolgous monkey or rhesus monkey, chimpanzees, or humans. In some embodiments, the subject is human.

“Therapeutically effective amount” or “effective amount” as used herein refers to an amount that is effective to elicit the desired biological or medical response, including the amount of a peptide that, when administered to a subject for treating a disease, is sufficient to effect such treatment for the disease. The effective amount will vary depending on the peptide, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered agents may optionally be lowered due to the combined action (e.g., additive or synergistic effects) of the additional agent and/or the peptide.

“Administer”, “administering”, or “administration” refers to delivering an amount of the peptide of the present invention to the subject.

“Co-administer”, “co-administering”, or “co-administration” refers to administration of unit dosages of the peptides disclosed herein before or after administration of unit dosages of one or more additional therapeutic agents, for example, administration of the peptide disclosed herein within seconds, minutes, or hours of the administration of one or more additional therapeutic agents. For example, in some embodiments, a unit dose of a peptide of the present disclosure is administered first, followed within seconds or minutes by administration of a unit dose of one or more additional therapeutic agents. Alternatively, in other embodiments, a unit dose of one or more additional therapeutic agents is administered first, followed by administration of a unit dose of a peptide of the present disclosure within seconds or minutes. In some embodiments, a unit dose of a peptide of the present disclosure is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a peptide of the present disclosure. Co-administration of a peptide disclosed herein with one or more additional therapeutic agents generally refers to simultaneous or sequential administration of a peptide disclosed herein and one or more additional therapeutic agents, such that therapeutically effective amounts of each agent are present in the body of the subject.

A voltage-gated sodium channel, “Nav channel”, or “Nav channel” is an integral membrane protein that forms an ion channel and conducts sodium ions through a plasma membrane in a cell. Nine known human sodium channel subtypes include hNav1.1, hNav1.2, hNav1.3, hNav1.4, hNav1.5, hNav1.6, hNav1.7, hNav1.8, and hNav1.9, wherein “hNav” or “hNav” indicates a human sodium channel. When referring to sodium channels from other species, such as rat (“rNav” or “rNav”) or mouse (“mNav” or “mNav”), the single letter prefix to “Nav” indicates the species as commonly understood in the art. For instance, rNav1.3 refers to a rat voltage-gated sodium channel subtype 1.3.

“Inhibit”, “inhibiting”, or “inhibition” refers to the actions of an agent to diminish or reduce the function of a biological target. Inhibitors include those that reduce the activity of a voltage-gated sodium channel.

### II. Peptides

In some embodiments, a peptide of the present invention is a peptide comprising Formula I:

(I)
(SEQ ID NO: 101)
X¹-X²-X³-K⁴-X⁵-X⁶-X⁷-X⁸-X⁹-D¹⁰-X¹¹-X¹²-R¹³-K¹⁴-
X¹⁵-X¹⁶-X¹⁷-G¹⁸-X¹⁹-R²⁰-X²¹-X²²-L²³-W²⁴-X²⁵-X²⁶-
X²⁷-X²⁸-X²⁹-X³⁰,

or a pharmaceutically acceptable salt thereof,
wherein
X¹ is Q, H, R, K, P, or Y;
X² is C or Sec;
X³ is Q or L;
X⁵ is W or A;
X⁶ is M, Nle, or F;
X⁷ is Q or W;
X⁸ is T or Q;
X⁹ is C or Sec;
X¹¹ is K, R, or S;
X¹² is D, A, T, S, or E;
X¹⁵ is C or Sec;
X¹⁶ is C or Sec;
X¹⁷ is E, D, A, or P;
X¹⁹ is F, norleucine (Nle), or L;
X²¹ is C or Sec;
X²² is R or norarginine (NorR);
X²⁵ is C or Sec;
X²⁶ is R or K;
X²⁷ is K or 2,4-dimethylphenylalanine (diMePhe);
X²⁸ is E or Q;
X²⁹ is L or tert-butylcysteine (tBuCys);
X³⁰ is L, A, C, D, E, F, G, H, I, K, M, N, P, Q, R, S, T, V, W, or Y, or is absent;
when X² and X¹⁶ are each C, the —SH groups between X² and X¹⁶ are combined to form a disulfide bond, or alternatively, X² and X¹⁶ each comprise a —SH group;
when X⁹ and X²¹ are each C, the —SH groups between X⁹ and X²¹ are combined to form a disulfide bond, or alternatively, X⁹ and X²¹ each comprise a —SH group;
when X¹⁵ and X²⁵ are each C, the —SH groups between X¹⁵ and X²⁵ are combined to form a disulfide bond, or alternatively, X¹⁵ and X²⁵ each comprise a —SH group; and
the C-terminus has a —C(O)NH₂, or alternatively, the C-terminus has a —C(O)OH.

In some embodiments of the peptide or a pharmaceutically acceptable salt thereof, X⁸ is T.

In some embodiments of the peptide or a pharmaceutically acceptable salt thereof, X¹⁷ is E.

In some embodiments, X² and X¹⁶ are each Sec. In some embodiments, the —SeH groups between X² and X¹⁶ are combined to form a diselenide bond. In some embodiments, X² and X¹⁶ each comprise a —SeH group.

In some embodiments, X⁹ and X²¹ are each Sec. In some embodiments, the —SeH groups between X⁹ and X²¹ are combined to form a diselenide bond. In some embodiments, X⁹ and X²¹ each comprise a —SeH group.

In some embodiments, X¹⁵ and X²⁵ are each Sec. In some embodiments, the —SeH groups between X¹⁵ and X²⁵ are combined to form a diselenide bond. In some embodiments, X¹⁵ and X²⁵ each comprise a —SeH group.

In some embodiments,
X² and X¹⁶ are each Sec, the —SeH groups between X² and X¹⁶ are combined to form a diselenide bond;
X⁹ and X²¹ are each Sec, the —SeH groups between X⁹ and X²¹ are combined to form a diselenide bond; and
X¹⁵ and X²⁵ are each Sec, the —SeH groups between X¹⁵ and X²⁵ are combined to form a diselenide bond.

In some embodiments, a peptide of the present invention is a peptide comprising Formula II:

(II)
(SEQ ID NO: 102)
X¹-C²-X³-K⁴-X⁵-X⁶-X⁷-T⁸-C⁹-D¹⁰-X¹¹-X¹²-R¹³-K¹⁴-
C¹⁵-C¹⁶-E¹⁷-G¹⁸-X¹⁹-R²⁰-C²¹-X²²-L²³-W²⁴-C²⁵-X²⁶-
X²⁷-E²⁸-X²⁹-X³⁰,

or a pharmaceutically acceptable salt thereof,
wherein
X¹ is Q, H, or Y;
X³ is Q or L;
X⁵ is W or A;
X⁶ is M, Nle, or F;
X⁷ is Q or W;
X¹¹ is K or S;
X¹² is D, A, or E;
X¹⁹ is F or L;
X²² is R or norarginine (NorR);
X²⁶ is R or K;
X²⁷ is K or 2,4-dimethylphenylalanine (diMePhe); and
X²⁹ is L or tert-butylcysteine (tBuCys).
   In some embodiments, X³⁰ is as defined herein.

In some embodiments of the peptide or a pharmaceutically acceptable salt thereof, the —SH groups between C² and C¹⁶ are combined to form a disulfide bond; the —SH groups between C⁹ and C²¹ are combined to form a disulfide bond; and the —SH groups between C¹⁵ and C²⁵ are combined to form a disulfide bond.

In some embodiments of the peptide or a pharmaceutically acceptable salt thereof, the C-terminus has a —C(O)NH₂.

In some embodiments of the peptide or a pharmaceutically acceptable salt thereof, the C-terminus has a —C(O)OH.

In some embodiments of the peptide or a pharmaceutically acceptable salt thereof, the peptide consists of Formula I or a pharmaceutically acceptable salt thereof.

In some embodiments of the peptide or a pharmaceutically acceptable salt thereof, the peptide consists of Formula II or a pharmaceutically acceptable salt thereof.

In some embodiments of the peptide or a pharmaceutically acceptable salt thereof, X¹ is Q or H. In some embodiments, X¹ is Q. In some embodiments, X¹ is H.

In some embodiments of the peptide or a pharmaceutically acceptable salt thereof, X³ is Q.

In some embodiments of the peptide or a pharmaceutically acceptable salt thereof, X⁵ is W.

In some embodiments of the peptide or a pharmaceutically acceptable salt thereof, X⁶ is M or Nle. In some embodiments, X⁶ is M. In some embodiments, X⁶ is Q.

In some embodiments of the peptide or a pharmaceutically acceptable salt thereof, X⁷ is Q.

In some embodiments of the peptide or a pharmaceutically acceptable salt thereof, X¹¹ is K.

In some embodiments of the peptide or a pharmaceutically acceptable salt thereof, X¹² is D.

In some embodiments of the peptide or a pharmaceutically acceptable salt thereof, X¹⁹ is F.

In some embodiments of the peptide or a pharmaceutically acceptable salt thereof, X²² is R.

In some embodiments of the peptide or a pharmaceutically acceptable salt thereof, X²⁶ is R.

In some embodiments of the peptide or a pharmaceutically acceptable salt thereof, X²⁷ is K.

In some embodiments of the peptide or a pharmaceutically acceptable salt thereof, X²⁹ is L.

In some embodiments of the peptide or a pharmaceutically acceptable salt thereof, X³⁰ is L, A, C, D, E, F, G, H, I, K, M, N, P, Q, R, S, T, V, W, or Y. In some embodiments, X³⁰ is absent. In some embodiments, X³⁰ is L, W, or Y. In some embodiments, X³⁰ is L. In some embodiments, X³⁰ is W. In some embodiments, X³⁰ is Y.

In some embodiments of the peptide or a pharmaceutically acceptable salt thereof, the peptide comprises Formula III:

(III)
(SEQ ID NO: 103)
X¹-C²-X³-K⁴-X⁵-X⁶-X⁷-T⁸-C⁹-D¹⁰-X¹¹-X¹²-R¹³-K¹⁴-
C¹⁵-C¹⁶-E¹⁷-G¹⁸-F¹⁹-R²⁰-C²¹-R²²-L²³-W²⁴-C²⁵-R²⁶-
K²⁷-E²⁸-L²⁹-L³⁰,

wherein X¹, X³, X⁵, X⁶, X⁷, X¹¹, and X¹² are as defined herein.

In some embodiments of the peptide or a pharmaceutically acceptable salt thereof, the peptide consists of Formula III or a pharmaceutically acceptable salt thereof.

In some embodiments, a peptide of the present invention is a peptide comprising Formula IV:

(IV)
(SEQ ID NO: 104)
X¹-C²-X³-X⁴-W⁵-M⁶-X⁷-Q⁸-C⁹-D¹⁰-X¹¹-X¹²-R¹³-X¹⁴-
C¹⁵-C¹⁶-X¹⁷-G¹⁸-L¹⁹-R²⁰-C²¹-R²²-L²³-W²⁴-C²⁵-R²⁶-
K²⁷-E²⁸-L⁻²⁹-X³⁰,

or a pharmaceutically acceptable salt thereof,
wherein
X¹ is Q, H, or R;
X³ is V, A, or L;
X⁴ is L, Y, K, N, or T;
X⁷ is Q or W;
X¹ is K or S;
X¹² is D or T;
X¹⁴ is K or R;
X¹⁷ is E or D;
X³⁰ is W or Y;
the —SH groups between C² and C¹⁶ are combined to form a disulfide bond; the —SH groups between C⁹ and C²¹ are combined to form a disulfide bond; and the —SH groups between C¹⁵ and C²⁵ are combined to form a disulfide bond; and the C-terminus has a —C(O)NH₂.

In some embodiments of the peptide or a pharmaceutically acceptable salt thereof, the peptide comprises the sequence:

(SEQ ID NO: 1)
QCQKWMQTCDKDRKCCEGFRCRLWCRKELL,
(SEQ ID NO: 2)
HCQKWMQTCDKDRKCCEGFRCRLWCRKELL,
(SEQ ID NO: 3)
HCQKW-Nle-QTCDKDRKCCEGFRCRLWCRKELL,
(SEQ ID NO: 4)
QCLKWMQTCDKDRKCCEGFRCRLWCRKELL,
(SEQ ID NO: 5)
HCQKWMQTCDKDRKCCEGFRCRLWCR-diMePhe-E-tBuCys-L,
(SEQ ID NO: 6)
QCQKAFQTCDKDRKCCEGFRCRLWCRKELL,
(SEQ ID NO: 7)
QCQKWMQTCDKARKCCEGFRCRLWCRKELL,
(SEQ ID NO: 8)
YCQKAFWTCDSERKCCEGLRC-NorR-LWCRKELW,
(SEQ ID NO: 9)
YCQKAFWTCDSARKCCEGLRC-NorR-LWCRKELW,
(SEQ ID NO: 10)
YCQKAFWTCDSARKCCEGLRCRLWCRKELW,
(SEQ ID NO: 11)
YCQKWMQTCDSARKCCEGLRCRLWCRKELW,
(SEQ ID NO: 12)
YCQKWMQTCDKDRKCCEGLRCRLWCRKELL,
(SEQ ID NO: 13)
QCQKWMQTCDSARKCCEGFRCRLWCRKELL,
or
(SEQ ID NO: 14)
YCQKAFWTCDSERKCCEGLRC-NorR-LWCKKELW.

In some embodiments, the peptide consists of any one of SEQ ID NOS: 1-14.

In some embodiments of the peptide or a pharmaceutically acceptable salt thereof, the peptide comprises the sequence:

(SEQ ID NO: 1)
QCQKWMQTCDKDRKCCEGFRCRLWCRKELL,
(SEQ ID NO: 2)
HCQKWMQTCDKDRKCCEGFRCRLWCRKELL,
or
(SEQ ID NO: 3)
HCQKW-Nle-QTCDKDRKCCEGFRCRLWCRKELL.

In some embodiments of the peptide or a pharmaceutically acceptable salt thereof, the peptide consists of the sequence:

(SEQ ID NO: 1)
QCQKWMQTCDKDRKCCEGFRCRLWCRKELL,
(SEQ ID NO: 2)
HCQKWMQTCDKDRKCCEGFRCRLWCRKELL,
or
(SEQ ID NO: 3)
HCQKW-Nle-QTCDKDRKCCEGFRCRLWCRKELL.

In some embodiments of the peptide or a pharmaceutically acceptable salt thereof, the peptide comprises the sequence:

(SEQ ID NO: 1)
QCQKWMQTCDKDRKCCEGFRCRLWCRKELL.

In some embodiments of the peptide or a pharmaceutically acceptable salt thereof, the peptide consists of the sequence:

(SEQ ID NO: 1)
QCQKWMQTCDKDRKCCEGFRCRLWCRKELL.

In some embodiments of the peptide or a pharmaceutically acceptable salt thereof, the peptide has sequence similarity to SEQ ID NO: 1:

(SEQ ID NO: 1)
QCQKWMQTCDKDRKCCEGFRCRLWCRKELL.

In some embodiments of the peptide or a pharmaceutically acceptable salt thereof, the peptide comprises the sequence:

(SEQ ID NO: 2)
HCQKWMQTCDKDRKCCEGFRCRLWCRKELL.

In some embodiments of the peptide or a pharmaceutically acceptable salt thereof, the peptide consists of the sequence:

(SEQ ID NO: 2)
HCQKWMQTCDKDRKCCEGFRCRLWCRKELL.

In some embodiments of the peptide or a pharmaceutically acceptable salt thereof, the peptide has sequence similarity to SEQ ID NO: 2:

(SEQ ID NO: 2)
HCQKWMQTCDKDRKCCEGFRCRLWCRKELL.

In some embodiments of the peptide or a pharmaceutically acceptable salt thereof, the peptide comprises the sequence:

(SEQ ID NO: 3)
HCQKW-Nle-QTCDKDRKCCEGFRCRLWCRKELL.

In some embodiments of the peptide or a pharmaceutically acceptable salt thereof, the peptide consists of the sequence:

(SEQ ID NO: 3)
HCQKW-Nle-QTCDKDRKCCEGFRCRLWCRKELL.

In some embodiments of the peptide or a pharmaceutically acceptable salt thereof, the peptide has sequence similarity to SEQ ID NO: 3:

(SEQ ID NO: 3)
HCQKW-Nle-QTCDKDRKCCEGFRCRLWCRKELL.

Sequence similarity may be quantitated by percent sequence identity. In some embodiments of the peptide or a pharmaceutically acceptable salt thereof, the peptide has a sequence identity of about 80%, 83%, 85%, 87%, 90%, 93%, 95%, 97%, or higher, to SEQ ID NO: 1. Percent identity can be determined for example by pairwise alignment using the default settings of the AlignX module of Vector NTI v.9.0.0 (Invitrogen, Carslbad, Calif.). The protein sequences of the present invention may be used as a query sequence to perform a search against public or patent databases, for example, to identify related sequences. Exemplary programs used to perform such searches are the XBLAST or BLASTP programs, or the GenomeQuest™ (GenomeQuest, Westborough, Mass.) suite using the default settings.

Pharmaceutically acceptable salts of peptides of the present invention include salts or zwitterionic forms of the peptides of the present invention which are water or oil-soluble or dispersible, which are suitable for treatment of diseases without undue toxicity, irritation, and allergic response, are commensurate with a reasonable benefit/risk ratio, and are effective for their intended use. The salts can be prepared during the final isolation and purification of the peptides or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Also, amino groups in the peptides of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. A pharmaceutically acceptable salt may suitably be a salt chosen, e.g., among acid addition salts and basic salts. Examples of acid addition salts include chloride salts, citrate salts and acetate salts.

Examples of basic salts include salts where the cation is selected among alkali metal cations, such as sodium or potassium ions, alkaline earth metal cations, such as calcium or magnesium ions, as well as substituted ammonium ions, such as ions of the type N(R1)(R2)(R3)(R4)+, where R1, R2, R3 and R4 independently will typically designate hydrogen, optionally substituted C₁₋₆-alkyl or optionally substituted C₂₋₆-alkenyl. Examples of relevant C₁₋₆-alkyl groups include methyl, ethyl, 1-propyl and 2-propyl groups. Examples of C₂₋₆-alkenyl groups of possible relevance include ethenyl, 1-propenyl and 2-propenyl. Other suitable base salts are formed from bases which form non-toxic salts. Representative examples include the aluminum, arginine, benzathine, calcium, choline, diethylamine, diolamine, glycine, lysine, magnesium, meglumine, olamine, potassium, sodium, tromethamine, and zinc salts. Hemisalts of acids and bases may also be formed, e.g., hemisulphate and hemicalcium salts.

Other examples of pharmaceutically acceptable salts are described in “Remington's Pharmaceutical Sciences”, 17^{th} edition, Alfonso R. Gennaro (Ed.), Mark Publishing Company, Easton, PA, USA, 1985 (and more recent editions thereof), in the “Encyclopaedia of Pharmaceutical Technology”, 3^{rd} edition, James Swarbrick (Ed.), Informa Healthcare USA (Inc.), NY, USA, 2007, and in J. Pharm. Sci. 66: 2 (1977). Also, for a review on suitable salts, see Handbook of Pharmaceutical Salts: Properties, Selection, and Use by Stahl and Wermuth (Wiley-VCH, 2002).

The peptides of the invention may be produced by chemical synthesis, such as solid phase peptide synthesis, on an automated peptide synthesizer. Alternatively, the peptides of the invention may be obtained from polynucleotides encoding the peptides by the use of cell-free expression systems such as reticulocyte lysate based expression systems, or by recombinant expression systems. Those skilled in the art will recognize other techniques for obtaining the peptides of the invention. The synthetic polynucleotide sequences encoding the peptides of the invention can be operably linked to one or more regulatory elements, such as a promoter and enhancer, that allow expression of the nucleotide sequence in the intended host cell. The synthetic polynucleotide may be a cDNA.

Further provided are isolated polynucleotides encoding the polypeptides described above, complements of the polynucleotides and equivalents of each thereof. In some embodiments, the polynucleotide is a DNA. In some embodiments, the polynucleotide is an RNA.

Generation of the peptides optionally having N-terminal and/or C-terminal extensions is typically achieved at the nucleic acid level. The polynucleotides may be synthesized using chemical gene synthesis according to methods described in U.S. Pat. Nos. 6,521,427 and 6,670,127, utilizing degenerate oligonucleotides to generate the desired variants, or by standard PCR cloning and mutagenesis. Libraries of variants may be generated by standard cloning techniques to clone the polynucleotides encoding the peptides into the vector for expression.

In some embodiments, the polynucleotides of the invention are produced by chemical synthesis such as solid phase polynucleotide synthesis on an automated polynucleotide synthesizer. Alternatively, the polynucleotides of the invention may be produced by other techniques such as PCR based duplication, vector based duplication, or restriction enzyme based DNA manipulation techniques. Techniques for producing or obtaining polynucleotides of known sequences are well known. In some embodiments, the polynucleotides of the invention can be codon optimized.

In some embodiments, a vector comprises the polynucleotide of the invention. Such vectors may be plasmid vectors, viral vectors, vectors for baculovirus expression, transposon based vectors or any other vector suitable for introduction of the polynucleotide of the invention into a given organism or genetic background by any means. For example, polynucleotides encoding the peptides of the invention are inserted into an expression vector and may be operably linked to control sequences in the expression vector to ensure efficient expression, such as signal sequences, promoters (e.g. naturally associated or heterologous promoters), enhancer elements, and transcription termination sequences, and are chosen to be compatible with the host cell chosen to express the peptides of the invention. Once the vector has been incorporated into the appropriate host, the host is maintained under conditions suitable for high level expression of the proteins encoded by the incorporated polynucleotides.

Suitable expression vectors are typically replicable in the host organisms either as episomes or as an integral part of the host chromosomal DNA. Commonly, expression vectors contain selection markers such as ampicillin-resistance, hygromycin-resistance, tetracycline resistance, kanamycin resistance or neomycin resistance to permit detection of those cells transformed with the desired DNA sequences.

Suitable promoter and enhancer elements are known in the art. For expression in a bacterial cell, suitable promoters include, but are not limited to, lacl, lacZ, T3, T7, gpt, lambda P and trc.

An exemplary vector for expression of the peptides is a vector having ampicillin-resistance selection marker, CMV promoter, CMV intron, signal peptide, neomycin resistance, fl origin of replication, SV40 polyadenylation signal, and cDNA encoding the peptide of the invention.

In some embodiments, a host cell comprises the vector of the invention. A “host cell” refers to any cell into which a vector has been introduced. It is understood that the term host cell is intended to refer not only to the particular subject cell but also to the progeny of such a cell. Because certain modifications may occur in succeeding generations due to either mutation or environmental influences, such progeny may not be identical to the parent cell, but are still included within the scope of the term “host cell” as used herein. Such host cells may be eukaryotic cells, prokaryotic cells, plant cells or archeal cells. Escherichia coli, bacilli, such as Bacillus subtilis, and other enterobacteriaceae, such as Salmonella, Serratia, and various Pseudomonas species, are examples of prokaryotic host cells.

Activity of a peptide of the present invention can be measured against hNav1.7 by any assay known in the art or described herein. For instance, the activity can be measured using a membrane depolarization assay using fluorescence resonance energy transfer (FRET) or a whole cell patch clamp assay. Exemplary assays to measure hNav1.7 activity include those described in U.S. Pat. Nos. 9,624,280, 9,279,003, 9,636,418, and 10344060, and US patent application publication nos. 20160222071, 20180105561, and 20180022786. Other assays include the in vitro and in vivo assays described in the Examples herein.

In some embodiments of the peptide or a pharmaceutically acceptable salt thereof, the peptide inhibits human Nav1.7. In some embodiments, the peptide has a human Nav1.7 IC₅₀ of less than about 10000 nM, less than about 1000 nM, less than about 100 nM, or less than about 10 nM in a patch clamp assay. In some embodiments, the peptide has a human Nav1.7 IC₅₀ of from about 0.1 nM to about 10000 nM, from about 1 nM to about 10000 nM, or from about 0.1 nM to about 5000 nM.

Selectivity of a peptide of the present invention for hNav1.7 against one or more other ion channels, such as a calcium, potassium, or sodium channel, can be measured by any assay known in the art or described herein. For instance, the selectivity can be measured by comparing IC₅₀ values from similar whole cell patch clamp assay results between hNav1.7 and another hNav channel. Exemplary assays to measure hNav1.7 selectivity include those described in U.S. Pat. Nos. 9,624,280, 9,279,003, 9,636,418, and 10344060, and US patent application publication nos. 20160222071, 20180105561, and 20180022786. Other assays include the in vitro and in vivo assays described in the Examples herein.

In some embodiments of the peptide or a pharmaceutically acceptable salt thereof, the peptide has a selectivity for human Nav1.7 over one or more of human Nav1.1, human Nav1.2, human Nav1.3, human Nav1.4, human Nav1.5, human Nav1.6, human Nav1.8, and/or human Nav1.9. In some embodiments of the peptide or a pharmaceutically acceptable salt thereof, the peptide has a selectivity for human Nav1.7 over human Nav1.2 and human Nav1.5. In an illustrative example, if a peptide has hNav1.7 IC₅₀=10 nM and hNav1.1 IC₅₀=1000 nM, the peptide has a selectivity for hNav1.7 of 100 over hNav1.1.

In some embodiments of the peptide or a pharmaceutically acceptable salt thereof, the peptide has a selectivity for human Nav1.7 over one or more of human Nav1.1, human Nav1.2, human Nav1.3, human Nav1.4, human Nav1.5, human Nav1.6, human Nav1.8, and/or human Nav1.9 of at least about 10, at least about 100, at least about 1000, or at least about 10000. In some embodiments, the peptide has a selectivity for human Nav1.7 over one or more of human Nav1.1, human Nav1.2, human Nav1.3, human Nav1.4, human Nav1.5, human Nav1.6, human Nav1.8, and/or human Nav1.9 of from about 3 to about 100000, from about 10 to about 100000, or from about 10 to about 10000.

In some embodiments of the peptide or a pharmaceutically acceptable salt thereof, the peptide has a selectivity for human Nav1.7 over human Nav1.1 of at least about 10, at least about 100, at least about 1000, or at least about 10000. In some embodiments, the peptide has a selectivity for human Nav1.7 over human Nav1.1 of from about 3 to about 100000, from about 10 to about 100000, or from about 10 to about 10000.

In some embodiments of the peptide or a pharmaceutically acceptable salt thereof, the peptide has a selectivity for human Nav1.7 over human Nav1.2 of at least about 10, at least about 100, at least about 1000, or at least about 10000. In some embodiments, the peptide has a selectivity for human Nav1.7 over human Nav1.2 of from about 3 to about 100000, from about 10 to about 100000, or from about 10 to about 10000.

In some embodiments of the peptide or a pharmaceutically acceptable salt thereof, the peptide has a selectivity for human Nav1.7 over human Nav1.3 of at least about 10, at least about 100, at least about 1000, or at least about 10000. In some embodiments, the peptide has a selectivity for human Nav1.7 over human Nav1.3 of from about 3 to about 100000, from about 10 to about 100000, or from about 10 to about 10000.

In some embodiments of the peptide or a pharmaceutically acceptable salt thereof, the peptide has a selectivity for human Nav1.7 over human Nav1.4 of at least about 10, at least about 100, at least about 1000, or at least about 10000. In some embodiments, the peptide has a selectivity for human Nav1.7 over human Nav1.4 of from about 3 to about 100000, from about 10 to about 100000, or from about 10 to about 10000.

In some embodiments of the peptide or a pharmaceutically acceptable salt thereof, the peptide has a selectivity for human Nav1.7 over human Nav1.5 of at least about 10, at least about 100, at least about 1000, or at least about 10000. In some embodiments, the peptide has a selectivity for human Nav1.7 over human Nav1.5 of from about 3 to about 100000, from about 10 to about 100000, or from about 10 to about 10000.

In some embodiments of the peptide or a pharmaceutically acceptable salt thereof, the peptide has a selectivity for human Nav1.7 over human Nav1.6 of at least about 10, at least about 100, at least about 1000, or at least about 10000. In some embodiments, the peptide has a selectivity for human Nav1.7 over human Nav1.6 of from about 3 to about 100000, from about 10 to about 100000, or from about 10 to about 10000.

In some embodiments of the peptide or a pharmaceutically acceptable salt thereof, the peptide has a selectivity for human Nav1.7 over human Nav1.8 of at least about 10, at least about 100, at least about 1000, or at least about 10000. In some embodiments, the peptide has a selectivity for human Nav1.7 over human Nav1.8 of from about 3 to about 100000, from about 10 to about 100000, or from about 10 to about 10000.

In some embodiments of the peptide or a pharmaceutically acceptable salt thereof, the peptide has a selectivity for human Nav1.7 over human Nav1.9 of at least about 10, at least about 100, at least about 1000, or at least about 10000. In some embodiments, the peptide has a selectivity for human Nav1.7 over human Nav1.9 of from about 3 to about 100000, from about 10 to about 100000, or from about 10 to about 10000.

For use in treating pain, a peptide should be stable under in vivo conditions for a period of time sufficient to provide the desired therapeutic effect. Accordingly, in some embodiments of the peptide or a pharmaceutically acceptable salt thereof, more than about 50% of the peptide is present after at least about 1 hour, at least about 2 hours, at least about 4 hours, at least about 8 hours, at least about 12 hours, at least about 1 day, at least about 2 days, at least about 3 days, at least about 4 days, or at least about 5 days, in cerebrospinal fluid. In some embodiments, from about 50% to about 90% of the peptide is present after at least about 1 hour, at least about 2 hours, at least about 4 hours, at least about 8 hours, at least about 12 hours, at least about 1 day, at least about 2 days, at least about 3 days, at least about 4 days, or at least about 5 days, in cerebrospinal fluid. In some embodiments, from about 50% to about 90% of the peptide is present after about 1 day in cerebrospinal fluid. In some embodiments, from about 50% to about 90% of the peptide is present after about 2 days in cerebrospinal fluid. In some embodiments, from about 50% to about 90% of the peptide is present after about 3 days in cerebrospinal fluid. In some embodiments, from about 50% to about 90% of the peptide is present after about 4 days in cerebrospinal fluid. In some embodiments, from about 50% to about 90% of the peptide is present after about 5 days in cerebrospinal fluid.

### III. Compositions

In some embodiments, the pharmaceutical composition of the present invention is a pharmaceutical composition comprising a peptide as described herein, or pharmaceutically acceptable salt thereof, and a pharmaceutically acceptable excipient.

### A. Formulation

For preparing pharmaceutical compositions from the peptide of the present invention, pharmaceutically acceptable carriers can be either solid or liquid. Solid form preparations include powders, cachets, and dispersible granules. A solid carrier can be one or more substances, which may also act as diluents, binders, preservatives, disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

The compositions of the present invention can be formulated for any suitable route of administration, including by one or more of oral, buccal, mucosal, sublingual, perenteral, subcutaneous, intramuscular, intraperitoneal, intrathecal, intranasal, inhalation, transdermal, rectal, or vaginal routes.

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the peptide of the present invention.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the peptide of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolality.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the peptides of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In another embodiment, the compositions of the present invention can be formulated for parenteral administration, such as intratumoral administration, intravitreal administration into an eye, or the intra-articular space of a joint. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV, intratumoral, or intravitreal administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46: 1576-1587, 1989).

Lipid-based drug delivery systems include lipid solutions, lipid emulsions, lipid dispersions, self-emulsifying drug delivery systems (SEDDS) and self-microemulsifying drug delivery systems (SMEDDS). In particular, SEDDS and SMEDDS are isotropic mixtures of lipids, surfactants and co-surfactants that can disperse spontaneously in aqueous media and form fine emulsions (SEDDS) or microemulsions (SMEDDS). Lipids useful in the formulations of the present invention include any natural or synthetic lipids including, but not limited to, sesame seed oil, olive oil, castor oil, peanut oil, fatty acid esters, glycerol esters, Labrafil®, Labrasol®, Cremophor®, Solutol®, Tween®, Capryol@, Capmul®, Captex®, and Peceol®.

### B. Administration

The peptides and compositions of the present invention can be delivered by any suitable means, including oral, parenteral and topical methods. In some embodiments, the delivery method is by one or more of oral, buccal, mucosal, sublingual, perenteral, subcutaneous, intramuscular, intraperitoneal, intrathecal, intranasal, inhalation, transdermal, rectal, or vaginal routes. In some embodiments, the delivery method is parenteral. In some embodiments, the delivery method is intrathecal. In some embodiments, the delivery method is intravenous. In some embodiments, the delivery method is subcutaneous.

The pharmaceutical preparation is preferably in unit dosage form. In such form the preparation is subdivided into unit doses containing appropriate quantities of the peptides and compositions of the present invention. The unit dosage form can be a packaged preparation, the package containing discrete quantities of preparation, such as packeted tablets, capsules, and powders in vials or ampoules.

Co-administration includes administering the peptide or composition of the present invention within 0.5, 1, 2, 4, 6, 8, 10, 12, 16, 20, or 24 hours of the other agent. Co-administration also includes administering simultaneously, approximately simultaneously (e.g., within about 1, 5, 10, 15, 20, or 30 minutes of each other), or sequentially in any order. Moreover, the peptides and compositions of the present invention can each be administered once a day, or two, three, or more times per day so as to provide the preferred dosage level per day.

In some embodiments, co-administration can be accomplished by co-formulation, i.e., preparing a single pharmaceutical composition including the peptides and compositions of the present invention and any other agent. Alternatively, the various components can be formulated separately.

The peptides and compositions of the present invention, and any other agents, can be present in any suitable amount, and can depend on various factors including, but not limited to, weight and age of the subject, state of the disease, etc. Suitable dosage ranges include from about 0.1 mg to about 10,000 mg, or about 1 mg to about 1000 mg, or about 10 mg to about 750 mg, or about 25 mg to about 500 mg, or about 50 mg to about 250 mg. Suitable dosages also include about 1 mg, 5, 10, 20, 30, 40, 50, 60, 70, 80, 90, 100, 200, 300, 400, 500, 600, 700, 800, 900 or 1000 mg. The composition can also contain other compatible therapeutic agents. The peptides described herein can be used in combination with one another, with other active agents known to be useful in modulating a glucocorticoid receptor, or with adjunctive agents that may not be effective alone, but may contribute to the efficacy of the active agent.

### IV. Methods of Use

In some embodiments, the method of the present invention is a method of inhibiting Nav1.7 in a cell, comprising administering to the cell an effective amount of a peptide as described herein, or a pharmaceutically acceptable salt thereof. In some embodiments, the cell is in vitro, ex vivo, or in vivo. In some embodiments, the cell is in vitro or ex vivo. In some embodiments, the cell is in vitro.

In some embodiments, the method of the present invention is a method of treating pain in a subject in need thereof, comprising administering to the subject a therapeutically effective amount of a peptide as described herein, or a pharmaceutically acceptable salt thereof.

Exemplary pain conditions include post-operative or post-traumatic pain, chronic lower back pain, pain of rheumatoid arthritis, osteoarthritis, fibromyalgia, cluster headaches, post-herpetic neuralgia, phantom limb pain, central stroke pain, dental pain, opioid-resistant pain, visceral pain, bone injury pain, labor pain, pain resulting from burns including sunburns, post-partum pain, migraine, tension type headache, angina pain, and genitourinary tract-related pain (e.g., cystitis). Types of pain include nociceptive pain, inflammatory pain, functional pain and neuropathic pain, which may be acute or chronic. Thus, the subject being treated may be diagnosed as having peripheral diabetic neuropathy, compression neuropathy, post herpetic neuralgia, trigeminal or glossopharyngeal neuralgia, post traumatic or post surgical nerve damage, lumbar or cervical radiculopathy, AIDS neuropathy, metabolic neuropathy, drug induced neuropathy, complex regional pain syndrome, arachnoiditis, spinal cord injury, bone or joint injury, tissue injury, psoriasis, scleroderma, pruritis, cancer (e.g., prostate, colon, breast, skin, hepatic, or kidney), cardiovascular disease (e.g., myocardial infarction, angina, ischemic or thrombotic cardiovascular disease, peripheral vascular occlusive disease, or peripheral arterial occlusive disease), sickle cell anemia, migraine cluster or tension-type headaches, inflammatory conditions of the skin, muscle, or joints, fibromyalgia, irritable bowel syndrome, non cardiac chest pain, cystitis, pancreatitis, or pelvic pain. Alternatively, the pain for which treatment is being sought may be the result of a traumatic injury, surgery, burn of the cutaneous tissue (caused by a thermal, chemical, or radiation stimulus), or a sunburn.

In some embodiments, the pain is chronic pain. For example, one type of chronic pain is neuropathic pain. In some embodiments, the chronic pain is continuous. In some embodiments, the chronic pain is intermittent. In some embodiments, the chronic pain is recurrent.

The therapeutically effective amount of the peptide of the invention, or a pharmaceutically acceptable salt thereof, may be administered by any suitable means in the art or described herein. In some embodiments, the method of the present invention comprises intrathecal, intravenous, or subcutaneous administration of the peptide or pharmaceutically acceptable salt thereof. In some embodiments, the method comprises intrathecal administration of the peptide or pharmaceutically acceptable salt thereof.

Treatment of pain refers to reducing or eliminating the sensation of pain in a subject before, during, or after the pain has occurred. As compared with an equivalent untreated control, such reduction or degree of prevention is at least 5%, 10%, 20%, 40%, 50%, 60%, 80%, 90%, 95%, or 100% as measured by any standard technique known in the art. To treat pain, according to the methods of this invention, the treatment may provide therapy for the underlying pathology that is causing the pain. Treatment of pain can be purely treatment of the pain symptoms.

Pain can be measured in a human subject by self-rating on different types of scales, including the numerical rating scale (NRS) and a visual analogue scale (VAS). Improvement of pain can be measured in the Patient Global Impression of Change (PGIC), the McGill Pain Questionnaire (SF-MPQ), the Brief Pain Inventory short form (BPI-SF), West Haven-Yale Multidimensional Pain Inventory (WHYMPI), or the Treatment Outcomes of Pain Survey (TOPS). See, Younger, J. et al. Curr. Pain Headache Rep. 2020, 13(1): 39-43.

### V. Examples

Abbreviations. Certain abbreviations and acronyms are used in describing the experimental details. Although most of these would be understood by one skilled in the art, Table 2 contains a list of many of these abbreviations and acronyms.

TABLE 2
List of abbreviations and acronyms.

| Abbre- | |
|---|---|
| viation | Meaning |
| aCSF | artificial cerebrospinal fluid |
| cryo-EM | cryogenic electron microscopy |
| DMEM | Dulbecco's Modified Eagle Medium |
| DODT | 3,6-dioxa-1,8-octanedithiol |
| DRG | dorsal root ganglion neuron |
| EGTA | ethylene glycol-bis(β-aminoethyl ether)-N,N,N′,N′-tetraacetic |
| | acid |
| HBSS | Hank's balanced salt solution or Hank's buffered saline |
| | solution |
| HEK-293 | human epithelial kidney-293 cell |
| HEPES | 4-(2-hydroxyethyl)-1-piperazineethanesulfonic acid |
| hERG | human ether-a-go-go-related gene (hERG) potassium channel |
| HPLC | high performance liquid chromatography |
| i.t. | intrathecal |
| LC/MS | liquid chromatography-mass spectrometry |
| MEM | minimum essential medium |
| OCT | optimal cutting temperature |
| PB | phosphate buffer |
| PDB | Protein Data Bank |
| TFA | trifluoroacetic acid |

The peptides described in the following Examples each display a C-terminus that has a —C(O)NH₂.

### Statistical Analysis

Results are expressed as means SEM. Statistical analysis was performed using Sigmaplot (version 14.0, Systat Software) or Igor Pro 8 (Wavemetrics). Results of in vitro experiments were analyzed using Student's t test (for differences between two groups). Results of in vivo experiments were analyzed using Two Way Repeated Measures ANOVA with Holm-Sidak post-hoc analysis. Differences between groups with P<0.05 were considered statistically significant. In experiments on mice technical replicates (n) were individual neurons and biological replicates (N) were individual mice. Details on statistical analysis are included in the figure legends. The sample power was calculated for rat behavioral studies with 8 animals per group is needed to show significant differences of 20% or more. The acceptable power level was considered to be between 0.8 and 0.9. For the thermal hyperalgesia test the mean value for the control population was assumed to be 7.5 seconds to distinguish a difference of 20% with a common standard deviation of about 10%. To test if the two populations are not equal at a significance level of 0.05, a power of 0.8 gives an n=8. The observed effect size was greater than expected and resulted in significant results with even smaller n. Investigators were blinded to identification of compound components in all studies. In brief, compound doses and vehicles were prepared and dosed on the day of the study by an independent researcher from those conducting the behavioral assessments. All treatment groups were randomized independent of baseline responses and the treatments included vehicle and positive controls were randomized on each day of assessment for blinded observers.

### Example 1. Design and Synthesis of ProTx-II Based Peptides Targeting hNav1.7

### Molecular Dynamics Simulation of ProTx-II—NavAb/Nav1.7 Chimera Complex.

A molecular dynamics simulation of the cryo-EM structure of NavAb/Nav1.7 in a complex with ProTx-II in a deactivated state (6N4R) was performed to obtain a closer look at the interaction of ProTx-II with lipid membrane at the residue level. CHARMM-GUI was used to embed the structure in a lipid bilayer of POPC with explicit TIP3P water molecules at a concentration of 150 mM NaCl. The system contained approximately 90,000 atoms and was parametrized with CHARMM36 forcefield. Neutral pH was used to assign the protonation state as default, and the C-terminus of ProTx-II was in the amidated form. The simulation was run on our local GPU cluster using NAMD version 2.12. After 10,000 steps of steepest descent minimization, 1 fs timestep equilibrations were started with harmonic restraints initially applied to protein-heavy atoms and lipid tail dihedral angles as suggested by CHARMM-GUI. These restraints were gradually released over 2 ns. Harmonic restraints (0.1 kcal/mol/A) were applied only to protein backbone heavy atoms. The systems were equilibrated further for 20 ns using 2 fs timestep with all bonds to hydrogen atoms constrained by the SHAKE algorithm. The equilibrations were performed in NPT ensemble with semi-isotropic pressure coupling to maintain the correct area per lipid, and a constant temperature of 303.15 K. Particle Mesh Ewald (PME) method was used to compute electrostatic interactions. Non-bonded pair lists were updated every 10 steps with a list cutoff distance of 16 Å and a real space cutoff of 12 Å with energy switching starting at 10 Å. The production run was conducted for 100 ns without applied protein backbone restraints.

The 100 ns simulation for interactions of ProTx-II residues with the surrounding environment was analyzed, categorized into different groups: lipid head, lipid tail, water and VSDII (hNav1.7/NavAb chimera). Fractional contact is defined as the frequency of forming contact (3.5 Å as a cutoff) of heavy atoms belonging to the associated groups normalized over the course of simulation and across interacting chains, A-E, B-F, C-G, D-H of the structure.

### Computational Design of ProTx-II Variants

First, the cryo-EM structure of ProTx-II in complex with hNav1.7/NavAb in a deactivated state (PDB: 6N4R) was further refined in Rosetta using Rosetta cryo-EM refinement protocol. Refined models (1000) were generated and the top 10 scoring models were extracted for visual inspection. How well ProTx-II fits into the electron density across multiple interacting chains A-E, B-F, C-G, and D-H of the top models was examined and eventually chain A-E was selected for the subsequent modeling.

Rosetta FastDesign was used to introduce ProTx-II substitutions and design new peptide variants. A small deviation of backbone conformation is inherently sampled in FastDesign by ramping cycles of reduced repulsive forces. Higher degrees of backbone flexibility during the design process were sought by further incorporating Rosetta Small mover and Roll mover. Small mover performs small random changes in the backbone torsional space while Roll mover invokes small rigid body perturbation between proTx-II and VSD-II. Both movers were implemented in Rosetta XML scripts prior to the FastDesign mover.

FastDesign was used in conjunction with sequence profile constraints to control amino acid identity substitutions. In the computational design step (round 2), fixed identity was applied to positions that reflect empirical knowledge such as R20, R22, E28 for preserving the hydrogen bond network with D816 (VSD-II) and W5, M6, W24, R26, K27, L29 for forming important interactions with the channel as observed from the modeling results of prior designs. On top of that, acidic residues were disallowed for positions that have significant with lipid head or lipid tail observed from the fractional contacts derived from the MD simulation of proTx-II—hNav1.7/NavAb chimera. Other positions, except disulfides were allowed to freely mutate. However, Rosetta Favor SequenceProfile mover was used to slightly bias new substitutions towards native residues on ProTx-II. This is due to the lack of secondary structural element on the majority of ProTx-II backbone in combination with using higher degree of backbone flexibility could result in less ideal amino acid substitutions with FastDesign. 1,000 designs were generated and the 100 top designs were extracted by total score followed by selecting top 20 designs by Rosetta DDG. The consensus designed sequence was constructed from the top 20 designs using sequence logo presentation. These top designs were analyzed to use in combination with established experimental data at different stages as described herein.

### Peptide Synthesis and Folding

The ProTx-II peptide variants were produced synthetically using Fmoc automated solid-phase synthesis performed on Liberty Blue peptide synthesizer from CEM Inc using a microwave assisted synthesis strategy employing diisopropyl carbodiimide and Oxyma for the activation chemistry. Pre-loaded ChemMatrix (Sigma Aldrich) Wang resins were used to produce ProTx-II variants with C-terminal acids. Acidolytic cleavage and deprotection of the completed peptide resins was performed with 9.5 ml trifluoroacetic acid (TFA), 0.5 ml H₂O, 0.5 ml Anisole, 0.5 ml thioanisole, 0.25 ml of DODT (3,6-dioxa-1,8-octanedithiol), 0.25 ml triisopropylsilane per gram of resin for 2 h at room temperature. Cleaved peptides were precipitated with 5-fold excess of diethyl ether added directly to the pre-filtered cleavage solution, isolated, and re-solubilized in TFA. Linear peptides were purified by preparative HPLC using a Phenomenex Luna C18(2), 100 Å pore size, 10 μparticle size, 250 mm×21.2 mm column and a 15-48% linear gradient of acetonitrile with 0.05% TFA over 40 min. Molecular weights were confirmed by LC/MS and fractions were pooled for folding. Purified linear fractions were added directly to 20 mM Tris, 2 M Urea, 1:2 oxidized/reduced glutathione, and pH was adjusted to 7.8-8.0 using acetic acid. Final peptide concentration was approximately 0.1-0.2 mg/ml. Solutions were stirred for 24-48 h at room temperature. Folded peptides were purified using a Phenomenex Luna C18(2), 100 Å pore size, 10 particle size, 250 mm×21.2 mm column with a 15-48% linear gradient of acetonitrile with 0.05% TFA over 40 min. Main peak fractions were analyzed by HPLC and LC/MS. Peptide fractions with a purity >95% were pooled, flash-frozen and subsequently lyophilized. Peptide content for each product was determined by absorbance at 280 nm using the calculated extinction coefficient. Percent purity was determined by HPLC using a Phenomenex Luna C18(2) analytical column, 250 mm×4.6 mm, 100 Å pore size, 5μ it particle size. Peptide mass and oxidation were confirmed by LC/MS using a Waters 2965 separations module coupled to a Waters Micromass ZQ electrospray mass spectrometer.

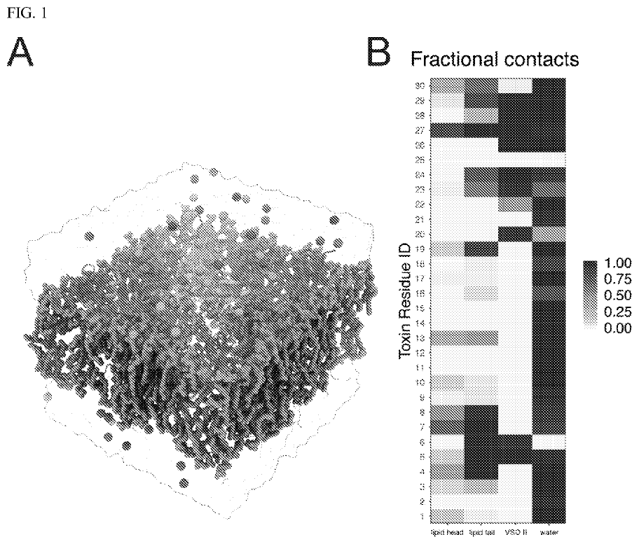

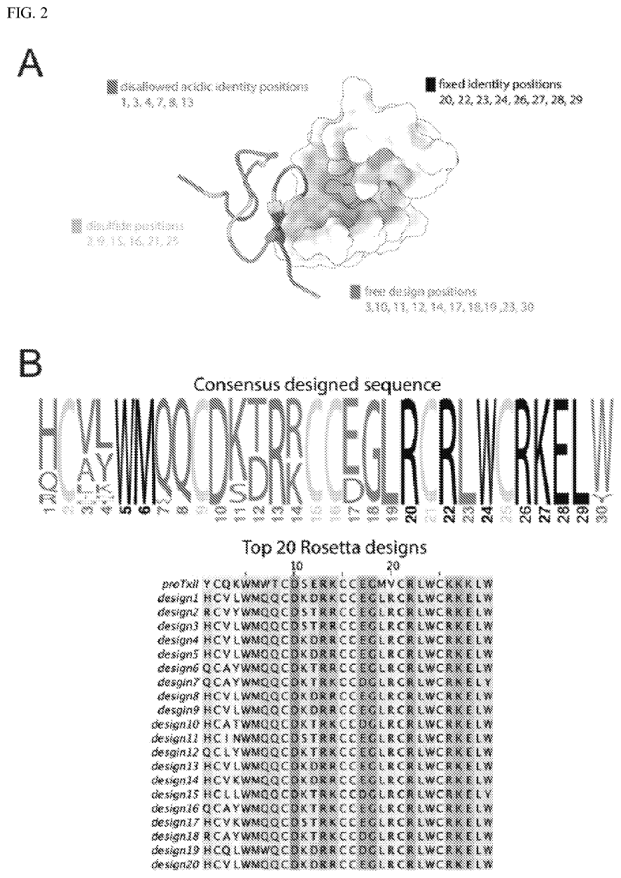

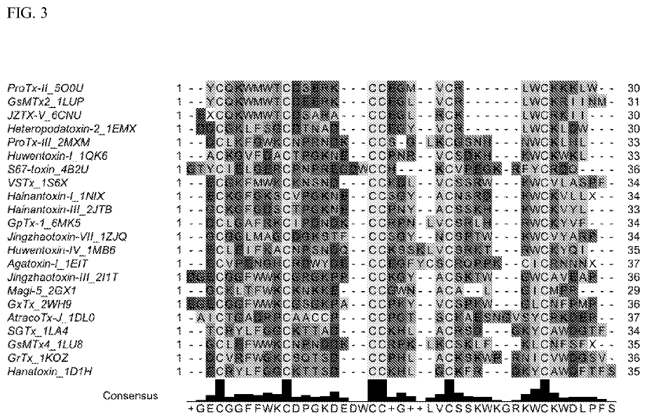

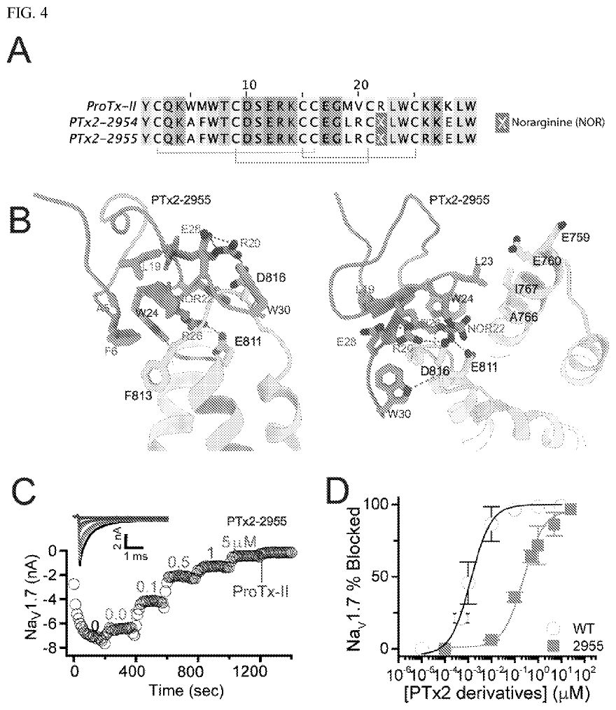

1st optimization round During the first round of optimization, multiple ProTx-II substitutions were guided by available experimental data and insights from the cryoEM structure of the ProTx-II—NavAb/hNav1.7 chimera complex in a deactivated state (PDB: 6N4R) (FIG. 1 and FIG. 2). The ProTx-II—NavAb/hNav1.7 structure revealed that ProTx-II residues W5 and M6 were positioned in the membrane hydrophobic core and made contact with the unique residue F813 on the S3 segment of hNav1.7 VSD-II (F812 in the NavAb/hNav1.7 structure) (FIG. 1). The W5A and M6F substitutions were introduced in ProTx-II with the insight from an Amgen's study showing that the double mutant F5A/M6F on GpTx-1 (ProTx-I homolog) improved selectivity for hNav1.7 over hNav1.4 and reasoning that optimized interactions with F813 may improve ProTx-II based peptide selectivity. In addition, the ProTx-II—NavAb/hNav1.7 structure revealed that the hydrophobic residue V20 was positioned in a hydrophilic environment and faced the hNav1.7 VSD-II S3-S4 loop region (FIG. 1). Based on the sequence comparison of ProTx-II to other highly potent peptide toxins targeting the hNav1.7 VSDII S3-S4 loop region (see FIG. 3), ProTx-III (SEQ ID NO: 44) (hNav1.7 IC₅₀=11.5 nM) had Lysine and JzTx-V (SEQ ID NO: 43) (hNav1.7 IC₅₀=0.6 nM) had Arginine at the position equivalent to the V20 in ProTx-II. Rosetta modeling of the ProTx-II V20R mutant suggested that arginine could form a salt bridge with D816 on the hNav1.7 VSD-II S3-S4 loop region (FIG. 4B). Because D816 is only present in the hNav1.7 and hNav1.6 subtypes among all human Nav channels (see FIG. 5), the V20R substitution was made to potentially improve selectivity for hNav1.7. A Genentech study demonstrated that substituting R22 with nor-arginine (norR) and K26 with arginine improves ProTx-II potency to below IC₅₀=0.1 nM for hNav1.7. Amgen's study demonstrated that substituting K28 with glutamate improves the selectivity of JzTx-V for hNav1.7 over Nav1.4 and Nav1.5. Based on these data, ProTx-IT R22 was substituted with norR, K26 with arginine, and K28 with glutamate. Also M19 was substituted with leucine to improve peptide stability to prevent methionine-dependent oxidation.

These substitutions were incorporated into two designed ProTx-II variants named PTx2-2954 (SEQ ID NO: 14) and PTx2-2955 (SEQ ID NO: 8). Specifically, PTx2-2954 (SEQ ID NO: 14) contained the W5A, M6F, M19L, V20R, R22norR, and K28E substitutions and PTx2-2955 (SEQ ID NO: 8) contained the W5A, M6F, M19L, V20R, R22norR, K26R, and K28E substitutions (FIG. 4A). The potency of PTx2-2954 and PTx2-2955 for hNav1.7 was determined using whole-cell voltage-clamp recordings in HEK 293 cells as described in Example 2. PTx2-2955 inhibited hNav1.7 currents with an IC₅₀ of 185.0 nM (FIGS. 4C and 4D). However, PTx2-2954 had no effect on hNav1.7 currents at 5 μM despite having only an arginine versus lysine difference at position 26 (FIG. 4A).

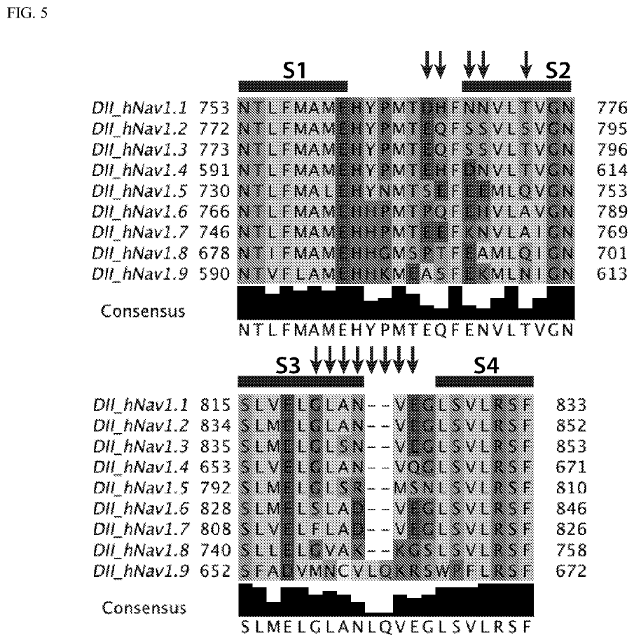

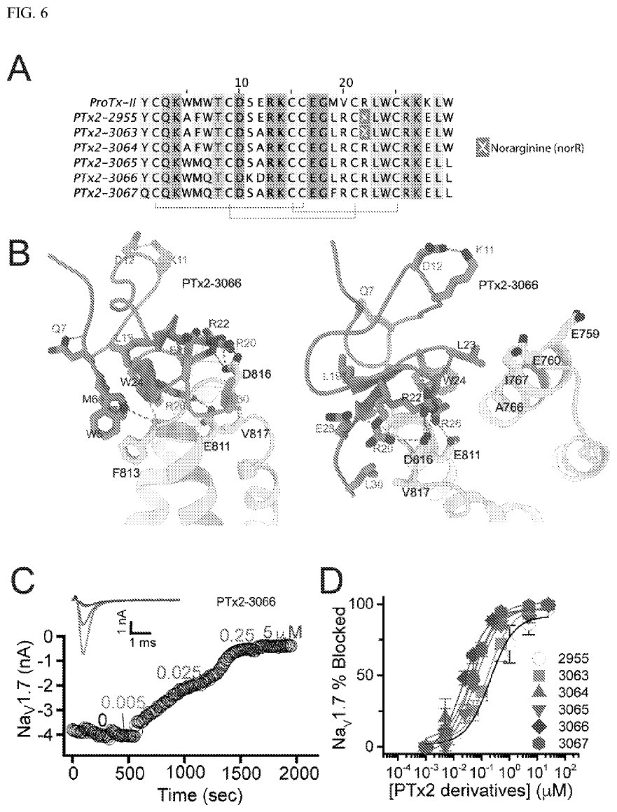

2nd optimization round. While the potency of PTx2-2955 (SEQ ID NO: 8) was not in the low nanomolar range, the molecular interactions revealed by computational modeling were useful for further rounds of optimization. Based on the modeling, R26 in PTx2-2955 had extensive contacts with VSD-II and formed a salt bridge with E811 (FIG. 4B). In addition, a hydrogen-bonding network was formed between residues R20, E28 on PTx2-2955 with D816 on VSD-II, a unique residue in hNav1.7 and hNav1.6 (FIG. 5). Such interactions were considered important for selectivity and given that the ProTx-II—VSD-II interface contained multiple polar contacts, room for further optimization of the molecular interface of ProTx-II and VSD-II may be limited. These interactions were preserved in this round of optimization and substitutions explored at other positions. Specifically, PTx2-3063 (SEQ ID NO: 9) was designed based on PTx2-2955 with an extra substitution E12A which was reported to improve the potency of ProTx-IT for hNav1.7. Notably, in the presence of R26, norarginine at position 22 did not appear to form a salt bridge with D816 on VSD-II despite being in proximity based on the PTx2-2955 model (FIG. 4B). The norarginine was mutated back to arginine to promote the hydrogen bond with D816 as it appeared in the wt ProTx-II and this was incorporated into the design PTx2-3064 (SEQ ID NO: 10). In the presence of R22, the hydrogen bond network at the interacting interface is expanded to E28, R20, and R22 on ProTx-II and D816 on VSDII (FIG. 6B). Rosetta computational design was further used to explore sequence variants at the non-interface positions of ProTx-II, explicitly looking for substitutions that can stabilize the ProTx-II scaffold or the interface hydrogen bond network while taking into account potential favorable interactions with lipids. The double mutants W5A/M6F were also changed back to the wild-type residues in the design process due to the lack of superior engagement with F813 (VSD-II) shown in the PTx2-2955 model. Among the consensus designed sequences suggested by Rosetta (see FIG. 2), the double mutant S11K/E12D and W7Q were selected to introduce in this round. S11K/E12D allowed a salt bridge to be formed between K and D while Q7 formed a hydrogen bond with a backbone carbonyl atom on ProTx-II, thus potentially stabilizing the ProTx-II scaffold and the hydrogen bond network between E28, R20, and R22 on ProTx-II and D816 on VSD-II. These substitutions were combined with other substitutions previously reported to improve potency or selectivity. In particular, Rosetta suggested substitution W7Q in addition to Y1Q, and W30L were shown to improve selectivity while M19F improved potency for hNav1.7. To reduce the potential of misfolding due to multiple substitutions, these changes were strategically introduced into three designed variants PTx2-3065 (SEQ ID NO: 11), PTx2-3066 (SEQ ID NO: 12), and PTx2-3067 (SEQ ID NO: 13).

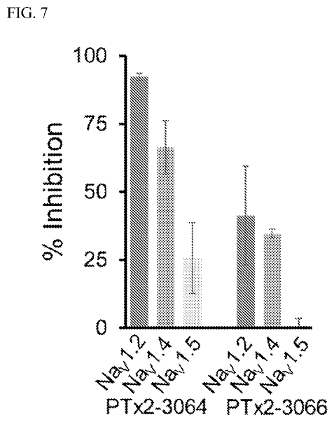

PTx2-3063 (SEQ ID NO: 9) and PTx2-3064 (SEQ ID NO: 10) peptides containing the same W5A and M6F mutations as PTx2-2955 (SEQ ID NO: 8) inhibited hNav1.7 currents with IC₅₀s of 154.0 and 52.6 nM, respectively (FIG. 6D). PTx2-3065 (SEQ ID NO: 11), PTx2-3066 (SEQ ID NO: 12), and PTx2-3067 (SEQ ID NO: 13) peptides containing the wild-type W5 and M6 residues inhibited hNav1.7 current with IC₅₀ values equal to 73.9, 30.8, and 48.3 nM, respectively (FIG. 6D). The selectivity of PTx2-3064 and PTx2-3066 peptides were tested for hNav1.7 versus other Nav channels (FIG. 7). PTx2-3064 and PTx2-3066 peptides blocked hNav1.2 current by ˜92 and ˜41% at 10 μM, respectively. PTx2-3064 and PTx2-3066 peptides blocked hNav1.5 current by ˜25 and ˜1% at 10 μM, respectively. PTx2-3064 and PTx2-3066 peptides blocked hNav1.4 current by ˜66% and ˜34% at 10 μM, respectively (FIG. 7).

3^{rd} optimization round. Building on the design of PTx2-3066 (SEQ ID NO: 12), other combinations were explored for Rosetta suggested substitutions and the reportedly improved potency/selectivity substitutions. Y lQ and M19F from the design of PTx2-3067 (SEQ ID NO: 13) were merged into PTx2-3066 with and without the double mutant W5A/M6F to generate new designs PTx2-3126 (SEQ ID NO: 6) and PTx2-3127 (SEQ ID NO: 1), respectively. In another design, PTx2-3128 (SEQ ID NO: 7), the scaffold stabilizing double mutant suggested by Rosetta, S11K/E12D, was explored for selectivity by introducing the potency improved substitution E12A which was used in the previous round.

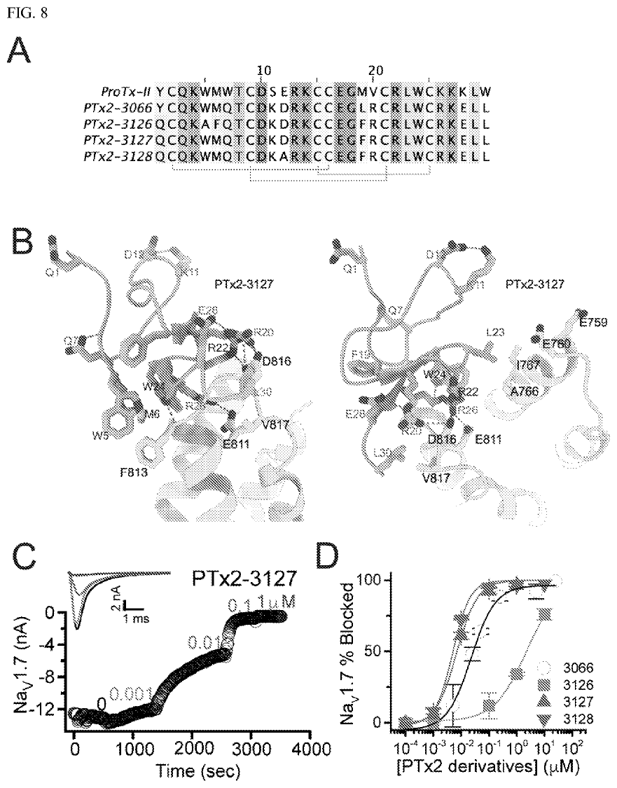

The PTx2-3126 (SEQ ID NO: 6) peptide containing the W5A and M6F mutations from PTx2-2955 and other mutations from PTx2-3066 inhibited hNav1.7 currents with an IC₅₀=2.3 μM (FIG. 8D). PTx2-3127 and PTx2-3128 containing the wild-type W5 and M6 residues and other mutations from PTx2-3066 inhibited hNav1.7 current with IC₅₀s equal to 6.9 and 5.0 nM, respectively (FIG. 8D). The selectivity of PTx2-3127 (SEQ ID NO: 1) and PTx2-3128 (SEQ ID NO: 8) were tested for hNav1.7 versus other Nav channels. PTx2-3127 inhibited Nav channels with the following IC₅₀ values: 17 μM (hNav1.1), 5 μM (hNav1.2), 20 μM (rNav1.3), 12 μM (hNav1.4), >130 μM (hNav1.5), 608 nM (hNav1.6), 7 nM (hNav1.7), >10 μM (hNav1.8), and 47 μM (hNav1.9). The data showed that PTx2-3127 was at least 1,000 fold selective for hNav1.7 versus hNav1.1, hNav1.3, hNav1.4, hNav1.5, hNav1.8, and hNav1.9. PTx2-3128 inhibited Nav channels with the following IC₅₀ values: 3.3 μM (hNav1.1), 570 nM (hNav1.2), 23 μM (rNav1.3), 22 μM (hNav1.4), 34 μM (hNav1.5), 358 nM (hNav1.6), 5 nM (hNav1.7), >10 μM (hNav1.8), and 8 μM (hNav1.9).

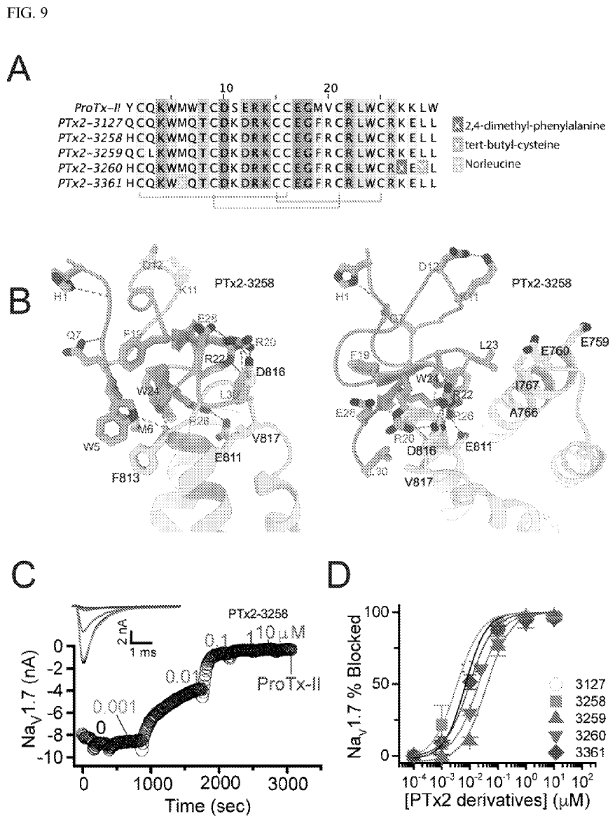

4th optimization round. Histidine appeared most frequently in the top Rosetta designs at position 1 (see FIG. 2). The structural model showed a hydrogen bond formed with a backbone carbonyl atom on ProTx-II (FIG. 9B) thus potentially stabilizing the ProTx-II scaffold. Building upon PTx2-3258 (SEQ ID NO: 2), Methionine at position 6 was replaced with Norleucine to prevent oxidation and incorporated the change in the design of PTx2-3361 (SEQ ID NO: 3). All previously tested substitutions selected by Rosetta were hydrogen bond promoted substitutions. In the design of PTx2-3259 (SEQ ID NO: 4), the Q3L substitution suggested by Rosetta (see FIG. 2) was tested whether it could create an additional stabilizing effect. The third most frequently observed amino acid at this position, Leu, was selected based on an experimental design protocol with the membrane scoring function. Lastly, non-canonical amino acids at positions 27 and 29 were explored to examine whether the selectivity of PTx2-3258 could be improved further given that these positions were near F813 (VSD-II). This resulted in the design of PTx2-3260 (SEQ ID NO: 5) with 2,4-dimethyl-phenylalanine and tert-butyl-cysteine at positions 27 and 29, respectively.

### Example 2. Potency and Selectivity Using Electrophysiological Assays on Recombinant Channel Cell Lines

HEK-293 cells stably expressing human Nav1.1, Nav1.4, Nav1.5, Nav1.6 and Nav1.7 were obtained from Dr. Chris Lossin. Rat Nav1.3 expressing HEK-293 cells were from Dr. Steven Waxman (Yale University, New Haven, CT). These cell lines were cultured in complete DMEM supplemented with 10% FBS, 1% penicillin/streptomycin, and G418. The human Nav1.8 channel (co-expressing with human Navβ1 and Navβ2 subunits) and the Nav1.9 channel (co-expressing with human Trkb, Navβ1, and Navβ2 subunits) were obtained from Icagen (Durham, NC). hNav1.8 cells cultured with G418 (0.4 mg/mL) and puromycin (0.5 ng/mL) and hNav1.9 cells were cultured with G418 (0.4 mg/mL), puromycin (0.5 ng/mL), and zeocin (0.05 mg/mL). Human Nav1.2 were expressed transiently by transfection of the hNav1.2 cDNA (from Dr. Alan L. Goldin, UC Irvine, CA) into HEK-293 cells.

Whole-cell patch-clamp experiments on recombinant channels were conducted manually at room temperature (22-24° C.) using an EPC-10 amplifier (HEKA Electronik, Lambrecht/Pfalz, Germany). Cells were trypsinized and plated onto poly-1-lysine-coated coverslips. All recordings were done in normal Ringer external bath solution containing (in mM) 160 NaCl, 4.5 KCl, 2 CaCl₂), 1 MgCl2, 10 HEPES (pH 7.4 and 305 mOsm) as. Patch pipettes were pulled from soda lime glass (micro-hematocrit tubes, Kimble Chase, Rochester, NY) and had resistances of 2-3 MQ when filled with CsF-based internal solution containing (in mM) 10 NaF, 110 CsF, 20 CsCl, 10 HEPES, 2 EGTA, (pH 7.4, 310 mOsm). Data acquisition and analysis were performed with Pulse-PulseFit (HEKA Electronik GmbH, Germany), IgorPro (WaveMetrics, Portland, OR), and Origin 9.0 software (OriginLab Corporation, Northampton, MA). Cells were held at −90 mV and voltage stepped to −120 mV for 200 ms before depolarizing to −10 mV for 50 msec to elicit inward currents. Control test currents were monitored for 5-10 min to ensure that the amplitude and kinetics of the response were stable. Series resistance was compensated to 80-90% and linear leak currents and capacitance artefacts were corrected using a P/4 subtraction method. Pulse interval was 0.1 Hz and peptides were applied to individual cells using a glass transfer pipette directly into the recording bath. For measuring inhibition, currents were allowed to saturate with repeated pulsing before addition of subsequent doses. IC₅₀ values were derived from measurements performed on individual cells that were tested with at least three or more concentrations of each peptide.

Functional characterization of the activity of the peptides PTx2-3258-PTx2-3260 and PTx2-3361 on the wild-type hNav1.7 expressed in HEK 293 cells analyzed by whole-cell voltage-clamp was performed. The ProTx-II variants inhibited the hNav1.7 channel with the following IC₅₀ values: PTx2-3158 (3.9 nM), PTx2-3259 (41.8 nM), PTx2-3260 (21.0 nM), and PTx2-3361 (9.0 nM). A summary of the human Nav1.7 activity of exemplary peptides of the invention is shown in Table 3 below.

TABLE 3
Peptide Activity against human Nav1.7

| PTx2 | SEQ ID NO: | hNav1.7 IC₅₀ (nM) |
|---|---|---|
| 3127 | 1 | 6.9 |
| 3258 | 2 | 3.9 |
| 3361 | 3 | 9 |
| 3259 | 4 | 41.8 |
| 3260 | 5 | 21 |
| 3126 | 6 | 2300 |
| 3128 | 7 | 5 |
| 2955 | 8 | 185 |
| 3063 | 9 | 154 |
| 3064 | 10 | 52.6 |
| 3065 | 11 | 73.9 |
| 3066 | 12 | 30.8 |
| 3067 | 13 | 48.3 |
| 2954 | 14 | >5000 |

Selectivity of certain peptides were tested across Nav subtypes. A summary of the data for PTx2-3127 (SEQ ID NO: 1), PTx2-3128 (SEQ ID NO: 7), and PTx2-3258 (SEQ ID NO: 2) are shown in Table 4 below.

TABLE 4
Human Nav Selectivity of Peptides.

| | PTx2-3127 | PTx2-3128 | PTx2-3258 |
|---|---|---|---|
| | (SEQ ID | (SEQ ID | (SEQ ID |
| Nav subtype | NO: 1) | NO: 7) | NO: 2) |
| hNav1.7 IC₅₀ (nM) | 6.9 | 5.0 | 3.8 |
| hNav1.1 IC₅₀ (nM) | 16,970 | 3300 | 5,013 |
| hNav1.2 IC₅₀ (nM) | 5,040 | 570 | 3,399 |
| rNav1.3 IC₅₀ (nM) | 20,040 | 23000 | 14,093 |
| hNav1.4 IC₅₀ (nM) | 11,530 | 22000 | 8,877 |
| hNav1.5 IC₅₀ (nM) | 137,090 | 34000 | 38,315 |
| hNav1.6 IC₅₀ (nM) | 608 | 358 | 382 |
| hNav1.8 IC₅₀ (nM) | >150,000 | 10000 | 43,079 |
| hNav1.9 IC₅₀ (nM) | >150,000 | 8000 | 59,443 |

Data denoted as the mean of individual IC₅₀'s (in nM) derived from recordings of 3 or more cells for each peptide.

The broader selectivity of PTx2-3127 (SEQ ID NO: 1) and PTx2-3258 (SEQ ID NO: 2) was tested on hERG channel. The ProTx-II variants inhibited the hERG channel with the following IC₅₀ values: PTx2-3127 (1.9 μM) and PTx2-3258 (1. μM). Therefore, PTx2-3127 has 272-fold and PTx2-3258 has 496-fold selectivity for hNav1.7 versus ERG channel. A summary is shown in Table 5 below, and comparison with exemplary peptides of the literature is shown in Table 6.

TABLE 5
Selectivity of PTx2-3127 and PTx2-3258

| | PTx2-3258 (SEQ ID NO: 2) | | PTx2-3127 (SEQ ID NO: 1) | |
|---|---|---|---|---|
| | | Selectivity | | Selectivity |
| | | for hNav1.7 | | for hNav1.7 |
| Cation | | vs Nav1.x | | vs Nav1.x |
| channel | IC₅₀ (nM) | (fold) | IC₅₀ (nM) | (fold) |
| hNav1.1 | 5,013 | 1,319 | 16,970 | 2,459 |
| hNav1.2 | 3,399 | 894 | 5,040 | 730 |
| rNav1.3 | 14,093 | 3,708 | 20,040 | 2,904 |
| hNav1.4 | 8,877 | 2,336 | 11,530 | 1,671 |
| hNav1.5 | 38,315 | 10,082 | 137,090 | 19,868 |
| hNav1.6 | 382 | 100 | 608 | 88 |
| hNav1.7 | 3.8 | 1 | 6.9 | 1 |
| hNav1.8 | 43,079 | 11,336 | >150,000 | >20,000 |
| hNav1.9 | 59,443 | 15,642 | >150,000 | >20,000 |
| hERG | 1,861 | 496 | 1,889 | 272 |

TABLE 6
Selectivity compared to other peptides.

| | | Selectivity | Selectivity | Selectivity | Selectivity |
|---|---|---|---|---|---|
| | hNav1.7 | for hNav1.7 | for hNav1.7 | for hNav1.7 | for hNav1.7 |
| | IC₅₀ | vs hNav1.2 | vs hNav1.4 | vs hNav1.5 | vs hNav1.6 |
| Peptide | (nM) | (fold) | (fold) | (fold) | (fold) |
| PTx2-3258 | 3.8 | 894 | 2,336 | 10,082 | 100 |
| (SEQ ID NO: 2) | | | | | |
| PTx2-3127 | 6.9 | 730 | 1,671 | 19,868 | 88 |
| (SEQ ID NO: 1) | | | | | |
| PTx2-3128 | 5.0 | 114 | 4,500 | 6,800 | 70 |
| (SEQ ID NO: 7) | | | | | |
| Janssen | 10 | 160 | 500 | >1,000 | 100 |
| JNJ63955918 | | | | | |
| ProTx-II | 0.3-1 | 100-140 | 260-380 | 300-1,000 | 86 |

### Example 3. Efficacy on Mouse Sensory Neurons

### Mice

This study conformed to guidelines established by the National Institutes of Health, Bethesda, MD. Mice were maintained on a 12 h light/dark cycle, and food and water was provided ad libitiim. The MrgprD^{GFP} mouse line was a generous gift from David Ginty (Harvard University, Boston, MA).

Preparation ofDRG Sections

MrgprD-GFP mouse line A 20-week-old mouse was briefly anesthetized with 3-5% isoflurane and then decapitated. The spinal column was dissected, and excess muscle tissue removed. The spinal column was then bisected in the middle of the L1 vertebrae identified by the 13^{th} rib and drop fixed for 1 hour in ice cold 4% paraformaldehyde in 0.1M phosphate buffer (PB) pH adjusted to 7.4. The spine was washed 3× for 10 min each in PB and cryoprotected at 4° C. in 30% sucrose diluted in PB for 24 hours. The spine was cut into sections containing two vertebra per sample which were frozen in Optimal Cutting Temperature (OCT) compound (Fisher Cat #4585) and stored at −80° C. until sectioning. Vertebrae position relative to the 13 rib was recorded for each frozen sample to determine the specific vertebrae position in the spinal cord. Samples were cut into 30 μm sections on a freezing stage sliding microtome and were collected on Colorfrost Plus microscope slides (Fisher Scientific Cat #12-550-19). Slides were stored at −20° C. or immediately used for multiplex immunofluorescence labeling.

### Multiplex Immunofluorescence Labeling

A hydrophobic barrier was drawn around tissue sections mounted on slides as described above using a hydrophobic barrier pen (Scientific Device Cat #9804-02). Sections were incubated in 4% milk in PB containing 0.2% Triton X-100 (vehicle) for 1 hour and then incubated in vehicle containing 0.1 mg/mL IgG F(ab) polyclonal IgG antibody (Abcam cat #ab6668) for 1 hour. Sections were washed 3× for 5 min each in vehicle and then incubated in vehicle containing primary Abs. for 1 hour. Sections were washed 3× for 5 min each in vehicle and then incubated in vehicle containing mouse IgG-subclass-specific goat secondary Abs conjugated to Alexa Fluor (Thermo Fisher). Sections were washed 3× for 5 min each in PB and mounted with Prolong Gold (Thermo Fisher) and Deckglaser cover glass (Cat #NC1776158). All incubations and washes were done at room temperature with gentle rocking.

### Immunofluorescence Imaging

Images were acquired with an inverted scanning imaging system (Zeiss LSM 880, 410900-247-075) run by ZEN black v2.1. Laser lines were 488 nm, 633 nm. Low-magnification images were acquired in confocal mode with a 0.8 NA 20× objective (Zeiss 420650-9901) and reconstructed as a tiled mosaic using ImageJ. High-magnification images were acquired in airy disk imaging mode with a 1.4 NA 63× oil objective (Zeiss 420782-9900-799). Linear adjustments to contrast and brightness and average fluorescence intensity z-projections were performed using ImageJ software.

### Neuron Cell Culture

Cervical, thoracic and lumbar DRGs were harvested from 4 to 6 week old MrprD-GFP mice and transferred to Hank's buffered saline solution (HBSS) (Invitrogen). Ganglia were treated with collagenase (2 mg/ml; Type P, Sigma-Aldrich) in HBSS for 15 min at 37° C. followed by 0.05% Trypsin-EDTA (Gibco) for 2.5 min with gentle rotation. Trypsin was neutralized with culture media (MEM, with L-glutamine, Phenol Red, without sodium pyruvate) supplemented with 10% horse serum (heat-inactivated; Gibco), 10 U/ml penicillin, 10 μg/ml streptomycin, MEM vitamin solution (Gibco), and B-27 supplement (Gibco). Serum-containing media was decanted and cells were triturated using a fire-polished Pasteur pipette in MEM culture media containing the supplements listed above. Cells were plated on laminin-treated (0.05 mg/ml, Sigma-Aldrich) 5 mm Deckglaser coverslips, which had previously been washed in 70% ethanol and UV-sterilized. Cells were then incubated at 37° C. in 5% CO₂. Cells were used for electrophysiological experiments 24-38 hours after plating.

### Voltage Clamp of Endogenous Neuronal Sodium Channels

Voltage clamp was achieved with a dPatch amplifier (Sutter Instruments) run by Sutterpatch (Sutter Instruments). Solutions for voltage clamp recordings: internal 15 mM NaCl, 100 mM CsCl, 25 mM CsF, 1 mM EGTA and 10 mM HEPES adjusted to pH 7.3 with CsOH, 297 mOsm. Seals and whole cell configuration were obtained in an external patching solution containing the following (in mM) 145 NaCl, 3.5 KCl, 1.5 CaCl₂), 1 MgCl2, 10 HEPES, 10 Gluscose adjusted to pH 7.4 with NaOH, 322 mOsm. For voltage clamp neuronal recordings, the external solution contained (in mM) 44 NaCl, 106 TEA-Cl, 1.5 CaCl₂), 1 MgCl2, 0.03 CdCl2 10 HEPES, 10 glucose, pH adjusted to 7.4 with TEA-OH, 315 mOsm. The calculated liquid junction potential for the internal and external recording solutions was 5.82 mV and not accounted for. Osmolality measured with a vapor pressure osmometer (Wescor, 5520). For voltage clamp recordings, neurons plated on cover glass as described in the Neuron Cell Culture section were placed in a recording chamber (Warner Cat #64-0381) and were rinsed with external patching solution using a gravity-driven perfusion system. Neurons from MrgprD^{GFP} mice showing intracellular GFP were then selected for patching. After whole cell voltage clamp was established the external patching solution was exchanged with the external recording solution using a gravity-driven perfusion system. PTx2-3127 (SEQ ID NO: 1), vehicle control (external recording solution) and TTX were kept on ice and diluted in room temperature (20-22° C.) external recording solution just prior to application to neurons and manually added at a rate of approximately 1 mL/min. Experimenter was blinded to identity of PTx2-3127 (SEQ ID NO: 1) versus vehicle control solutions during recordings. PTx2-3127 (SEQ ID NO: 1), vehicle control and TTX were applied to neurons using separate perfusion lines to prevent contamination. After each neuron, perfusion lines were cleared with 1 mL of 70% ethanol followed by 1 mL of milli Q water and were then filled with external recording solution. Thin-wall borosilicate glass recording pipettes (BF150-110-10, Sutter) were pulled with blunt tips, coated with silicone elastomer (Sylgard 184, Dow Corning), heat cured, and tip fire-polished to resistances less than 3 MΩ. Series resistance of 3-8 MΩ was estimated from the whole-cell parameters circuit. Series resistance compensation between 37-77% was used to constrain voltage error to less than 15 mV, lag was 6 ρs. Cell capacitances were 13-34 pF. Capacitance and Ohmic leak were subtracted using a P/4 protocol. Output was low-pass filtered at 10 kHz using the amplifier's built-in Bessel and digitized at 50 kHz. The average current in the initial 0.14 seconds at holding potential prior to the voltage step was used to zero-subtract each recording. Mean current was the current amplitude between 0.4-1 ms into the 0 mV step. Peak current amplitude was the peak current amplitude between 0.4-8 ms into the 0 mV step. Experiments were performed on neurons with membrane resistance greater than 1 GQ assessed prior to running voltage clamp or current clamp protocols while neurons were held at a membrane potential of −80 mV. Data with predicted voltage error, Vₑᵣᵣₒᵣ≥15 mV were excluded from analysis. Vₑᵣᵣₒᵣ was tabulated using estimated series resistance post compensation and peak Nav current.

### Current Clamp

Solutions for current clamp recordings: internal (in mM) 120 K-methylsulfonate, 10 KCl, 10 NaCl, 5 EGTA, 0.5 CaCl₂), 10 HEPES, 2.5 MgATP and adjusted to pH 7.2, 289 mOsm. External solution (in mM) 145 NaCl, 5 KCl, 2 CaCl₂), 2 MgCl2, 10 HEPES, 10 Glucose adjusted to pH 7.3 with NaOH, 308 mOsm. The calculated liquid junction potential for these solutions was 9.682 mV and not accounted for. Thin-wall borosilicate glass recording pipettes (BF150-110-10, Sutter) were pulled with blunt tips and tip fire-polished to resistances less than 3 Mn. For current clamp recordings, neurons plated on cover glass as described in the Neuron Cell Culture section were placed in a recording chamber (Warner Cat #64-0381) and were rinsed with external solution using a gravity-driven perfusion system. Neurons from MrgprD-GFP mice showing intracellular GFP were then selected for patching. The same protocol for application of PTx2-3127 (SEQ ID NO: 1), vehicle control (external solution) and TTX described in the Voltage Clamp section was followed. In current clamp experiments data were excluded if the resting membrane potential of a neuron rose above −40 mV.

### Experimental Design and Statistical Treatment

Independent replicates (n) were individual neurons from multiple mice, details in figure legends. Statistical tests were conducted using Igor 8 (Wavemetrics Inc), details in figure legends.

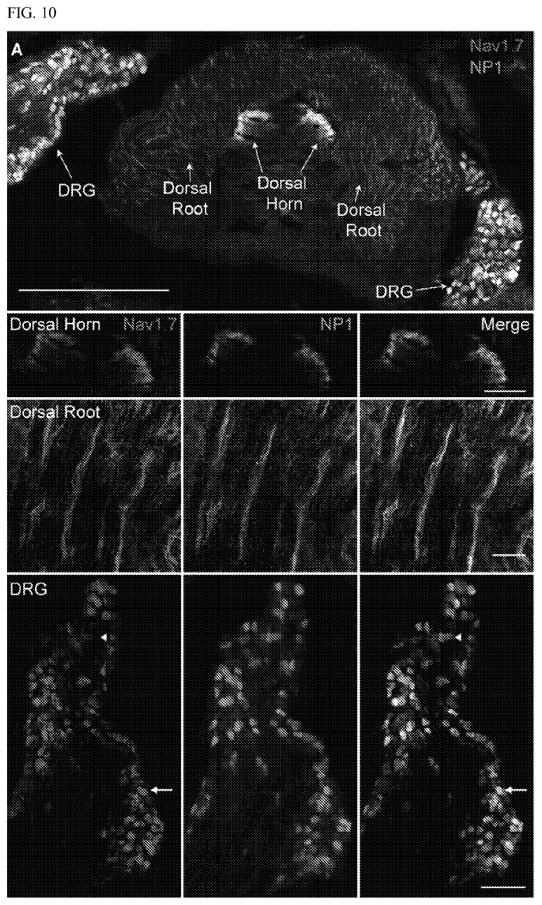

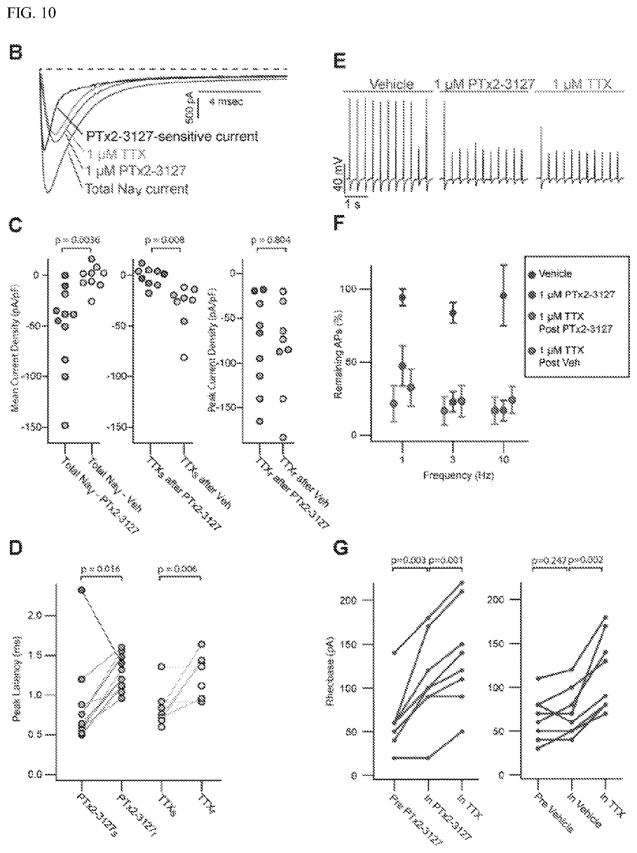

Nav1.7 is believed to be important for pain signaling in mice. As mice are valuable preclinical models for therapeutic development it is important to know whether mouse endogenous Nav1.7 is responsive to any therapeutic candidate. The effects of PTx2-3127 (SEQ ID NO: 1) on Nav currents of genetically-identified mouse nociceptor sensory neurons were studied. MrgprD⁺ nonpeptidergic nociceptors were identified by fluorescence in MrgprD^{GFP} mice. MrgprD^{GFP} DRG neurons from adult mice have significant expression of mRNA for Naᵥ1.7, Naᵥ1.8 and Naᵥ1.9 with other Nav transcripts in much lower abundance (Naᵥ1.8˜Naᵥ1.9>Naᵥ1.7>>Naᵥ1.6>>Naᵥ1.1). Presence of Nav1.7 protein in DRG neurons of the MrgprD^{GFP} mouse line used for electrophysiology was confirmed by observation of anti-Nay1.7 immunofluorescence in MrgprD^{GFP} DRG neuron cell bodies and axonal processes (FIG. 10A), consistent with prior reports of Nav1.7 localization to small, unmyclinated neurons. Anti-Nav1.7 immunofluorescence was variable in MrgprD^{GFP} DRG neurons with some exhibiting high and others low density of Nav1.7 protein (FIG. 10A).

Application of 1 μM PTx2-3127 (SEQ ID NO: 1) to dissociated MrgprD^{GFP} neurons under voltage clamp resulted in elimination of a fast-inactivating Nav component (FIG. 10B black trace). Blinded, interleaved experiments with either 1 μM PTx2-3127 or vehicle revealed that PTx2-3127 inhibited 48±17 pA/pF (mean SEM) of inward current 0.4-1 ms into a 0 mV step, while vehicle had little effect, inhibiting 2±6 pA/pF (FIG. 10C, left). Subsequent application of 1 μM TTX to the vehicle controls inhibited 37±12 pA/pF of inward current, similar to the density inhibited by PTx2-3127. Subsequent application of 1 μM TTX to PTx2-3127 had had little effect, 2.1±2.8 pA/pF, showing PTx2-3127 inhibits TTX-sensitive currents in MrgprD^{GFP} neurons (FIG. 10C, middle). The density of inhibitor-resistant peak current was similar for TTX PTx2-3127 (FIG. 10C, right). Comparison of Nav current peak times substantiated the observation that PTx2-3127-sensitive currents were faster than PTx2-3127-resistant currents (FIG. 10D). In vehicle controls TTX-sensitive peak currents were faster than TTX-resistant peak currents, consistent with a prior study of MrgprD^{GFP} neurons. Overall, the similar effects of either PTx2-3127 or TTX on Nav currents suggests PTx2-3127 targets the TTX-sensitive channels of MrgprD^{GFP} neurons. MrgprD^{GFP} neurons express Nav1.7, which is TTX-sensitive, and have much lower transcript abundances of the other TTX-sensitive channels, Nav1.1, 1.2, 1.3, 1.4, 1.6. Thus, the properties of PTx2-3127 were consistent with the peptide inhibiting Nav1.7 channels in mouse MrgprD⁺ nociceptors.

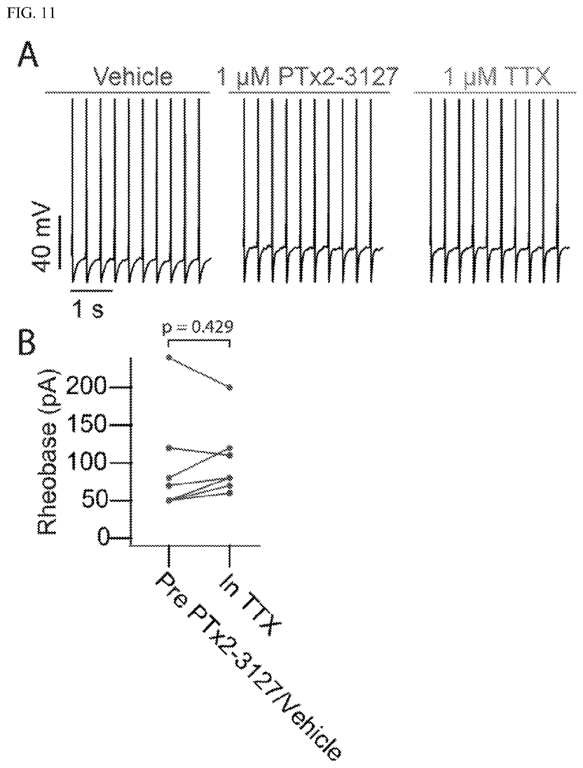

The impact of the designed peptide on action potential firing of dissociated MrgprD^{GFP} neurons was assessed with current-clamp recording. Action potentials were recorded in vehicle, then 1 μM PTx2-3127 (SEQ ID NO: 1), then 1 μM TTX. Blinded interleaved controls were conducted with vehicle replacing PTx2-3127. Rheobase, the step current required to evoke a single action potential, was increased by PTx2-3127 (FIG. 10G). When stimulated with 20 ms current injections at 150% of rheobase at 1, 3, and 10 Hz, PTx2-3127 suppressed repetitive firing of most neurons (FIG. 10E). In 27% of neurons, no block of action potentials was observed by TTX (FIG. 11), and these TTX-insensitive neurons were not included in further analyses. In all of the TTX-sensitive neurons, action potentials were blocked by PTx2-3127, and subsequent application of TTX had little additional effect (FIG. 10F). Our data demonstrated that PTx2-3127 was effective at inhibiting nociceptor excitability and action potentials in a mouse neuron model.

### Example 4. Efficacy on Human Sensory Neurons

All human tissues that were used for the study were obtained by legal consent from organ donors in the US. AnaBios Corporation's procurement network includes only US based Organ Procurement Organizations and Hospitals. Policies for donor screening and consent are the ones established by the United Network for Organ Sharing (UNOS). Organizations supplying human tissues to AnaBios follow the standards and procedures established by the US Centers for Disease Control (CDC) and are inspected biannually by the DHHS. Distribution of donor medical information is in compliance with HIPAA regulations to protect donor's privacy. All transfers of donor tissue to AnaBios are fully traceable and periodically reviewed by US Federal authorities. AnaBios generally obtains donor organs/tissues from adults aged 18 to 60 years old. Donor DRGs from males and females were harvested using AnaBios' proprietary surgical techniques and tools and were shipped to AnaBios via dedicated couriers. The DRGs were then further dissected in cold proprietary neuroplegic solution to remove all connective tissue and fat. The ganglia were enzymatically digested, and the isolated neurons put in culture in DMEM F-12 (Gemini Bio-Products CAT #: 900-955. Lot #M96R00J) supplemented with Glutamine 2 mM, Horse Serum 10% (Invitrogen #16050-130), hNGF (25 ng/ml) (Cell Signaling Technology #5221LF), GDNF (25 ng/ml) (ProSpec Protein Specialist #CYT-305) and Penicillin/Streptomycin (Thermo Fischer Scientific #15140-122).

External Current Clamp solution included: 145 mM NaCl, 3 mM KCl, 1 mM MgCl2, 2 mM CaCl₂), 10 mM dextrose, 10 mM HEPES, pH=7.4 (with NaOH), 300±5 mOsm. Internal Current Clamp solution included: 110 mM K+Gluconate, 20 mM KCl, 10 mM EGTA, 8 mM NaCl, 4 mM Mg-ATP, 10 mM HEPES, pH=7.3 (with KOH), 280±5 mOsm. All of our compounds come from Sigma-Aldrich. PTx2-3127 (SEQ ID NO: 1) was stored in 10 mM formulation in DMSO at −20° C. Oxaliplatin was stored in 50 mM formulation in DMSO at 4° C.

DRG recordings were obtained from human DRG in culture (2 to 7 days). Human DRG neurons were incubated with Oxaliplatin (50 μM) at 37° C. for 24 h. Whole-cell patch-clamp recordings were conducted under current-clamp mode at room temperature (˜23° C.) using HEKA EPC-10 amplifier. Data were acquired on a Windows-based computer using the PatchMaster program. Pipettes (1.5-3.0 MA) (Warner Instruments #64-0792) were fabricated from 1.5 mm borosilicate capillary glass using a Sutter P-97 puller. Cells on Corning glass coverslips (Thomas Scientific #354086) were transferred to a RC-26GLP recording chamber (Warner Instruments #64-0236) containing 0.5 ml standard external solution. Extracellular solution exchange was performed with rapid exchange perfusion system (flow rate 0.5-1 ml/min) (Warner Instruments #64-0186). Cells for recordings were selected based on smoothness of the membrane. Cells were held at a resting membrane potential. Signals were filtered at 3 kHz, sampled at 10 kHz. Once whole-cell access was obtained the cell was allowed an equilibration time of at least 5 min. Once the cell under recording stabilized, rheobase of single action potentials were assessed. Action potentials were induced by a train of 10 individual current steps 20 ms in. duration, delivered at 0.1 Hz and 120 individual current steps delivered at 1, 3 and 10 Hz, using current injection at 150% of rheobase of baseline. Test compound concentrations were washed in for 5 minutes and step 6 and 7 were repeated for each concentration. Exclusion criteria: series resistance >15 MΩ; unstable recording configuration (15% change of rheobase or access resistance within the same concentration); time frame of drug exposure not respected.

The percentage of action potentials remaining was calculated as the number of action potentials divided by the number of action potentials obtained under control condition at the same frequency. One-way ANOVA (SigmaPlot v14) with Tukey, Bonferroni and Dunnett post-hoc test was used to determine the significance of difference between treatment and control.

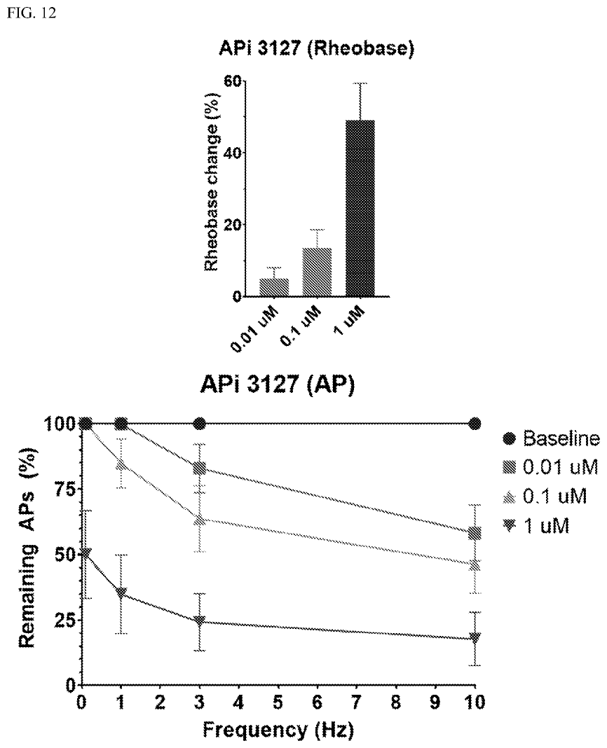

The effects of PTx2-3127 (SEQ ID NO: 1) were studied on the inhibition of single and multiple action potentials properties generated in adult human DRG neurons isolated from a human organ donor. The DRG neurons in culture were treated for 24 hrs. with 50 μM oxaliplatin to model chemotherapy-induced neuropathy. Rheobase was found to increase with increasing concentrations of PTx2-3127 (FIG. 12, upper graph). Action potentials were then measured, induced by a train of 10 to 120 individual current steps delivered at 0.1, 1, 3, and 10 Hz, using current injection at 150% of baseline rheobase. The percentage of action potentials remaining was calculated as the number of action potentials in the presence of PTx2-3127 divided by the number of action potentials obtained under control conditions (without drug) at the same frequency. The number of remaining action potentials was reduced in a dose-dependent manner at 0.01, 0.1, and 1 μM PTx2-3127 at different frequencies following 24 hours of incubation with Oxaliplatin (FIG. 12, lower graph). PTx2-3127 was effective at inhibiting human sensory neurons' excitability and action potentials in an in vitro model of chemotherapy-induced neuropathy.

### Example 5. Stability in Artificial Cerebrospinal Fluid

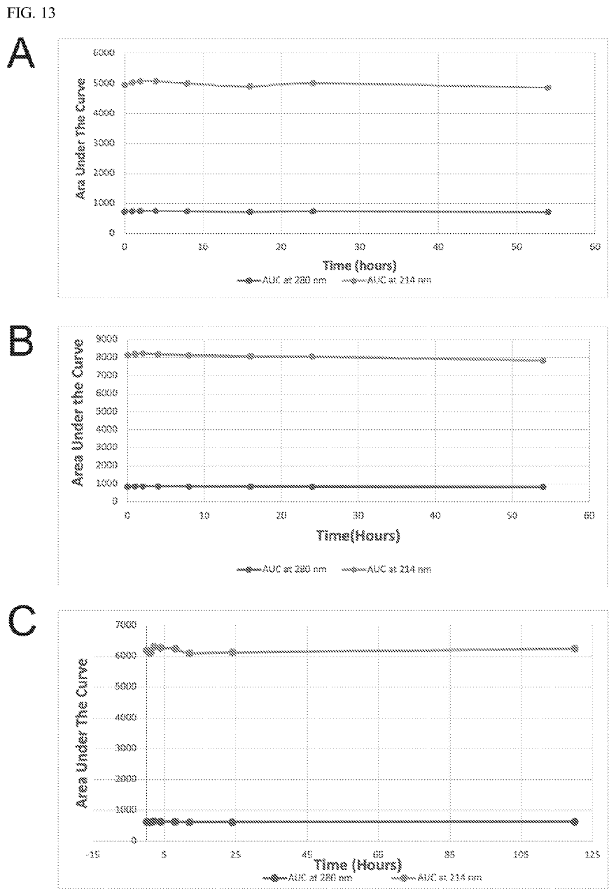

Stability In Artificial Cerebrospinal Fluid (aCSF): The stability of Native Protoxin, PTx2-3127, PTx2-3258, and PTx2-3361 was conducted in artificial Cerebrospinal Fluid (aCSF). The aCSF was purchased from Tocris Biosciences (Catalog #3525) and had the following ion composition (in mM): Na⁺ 150; K⁺ 3.0; Ca²⁺ 1.4; Mg²⁺ 0.8; P 1.0; Cl⁻ 155. Native Protoxin II, PTx2-3127 (SEQ ID NO: 1), and PTx2-3258 (SEQ ID NO: 2) were dissolved in DPBS at 200 μM (1 mg of respective peptides in 1305 mL, 1315 mL, and 1315 mL of DPBS respectively). 500 mL of dissolved peptide in DPBS and 1500 mL of aCSF were mixed to get 50 μM peptide solution in aCSF. The samples were incubated at 37° C. and aliquots of 100 μL were removed at 0, 1 hrs, 2 hrs, 4 hrs, 8 hrs, 12 hrs, 24 hrs, and 120 hrs respectively. The aliquots were immediately flash frozen and stored at −80° C. until further analysis. Peptides dissolved in aCSF were analyzed on an Agilent HPLC system (Please ask Vikrant for model) and monitored at 214 nm and 280 nm. The stability at various time points was determined by calculating the average Area under the curve at 214 nm and 280 nm for 2 injections of 20 μL using ChemStation Software. The peptides were run on a BioBasic™ C18 column (150×4.8 mm, ThermoFisher). The mobile phases were 0.1% Trifluoroacetic Acid in water (mobile phase A) and 100% Acetonitrile (mobile phase B). The results are shown in FIG. 13A-13C.

To study our lead peptide stability in artificial cerebrospinal fluid, a stock solution of PTx2-3127 peptide (SEQ ID NO: 1) at 200 μM was prepared. The peptide was dissolved in aCSF (Tocris, Catalog number 3525) to a final concentration. A standard curve for the peptide was made and then a stability assay was conducted at various time points. PTx2-3127 was stable in artificial CSF at 37° C. for more than 50 hours (FIG. 13B).

### Example 6. Efficacy in Animal Models of Pain

### Animals

All experiments using live animals were conducted in accordance with protocols approved by the Institutional Animal Care and Use Committee of the University of California and adhered to the National Institutes of Health guide for the care and use of Laboratory animals. Great care was taken to reduce the number and minimize suffering of the animals used. Sprague-Dawley male and female rats (250 to 300 g; Charles River, Wilmington, MA, USA) were housed with free access to food and water. They were maintained under a 12 h light/dark cycle with controlled temperature and relative humidity. After acclimation, the animals were each assayed for their baseline responses and then a day later received an intrathecal port placement. After recovery from the port surgery, the rats were assessed for post-surgery behavioral testing. For peptide treatments, rats were randomly divided into groups and tested with assays performed between 9:00 a.m. and 5:00 p.m. Scientists running the experiments were blinded to the treatment protocol at the time of the tests.

For the intrathecal cannulation briefly, the rats were anesthetized by isoflurane inhalation and the hair on the back at the surgical site shaved and the skin cleaned with ethyl alcohol and betadine per aseptic technique and incised about 1 cm in length. The muscle on the side of the L4-L5 vertebrae was incised and retracted to place a catheter into the subarachnoid space. The tissue was incised by the tip of a bent needle, which allows escape of a small amount of cerebral spinal fluid (CSF). The caudal edge of the cut is lifted, and an intrathecal catheter, 32ga (0.8Fr) PU 18 cm, fixed to a stylet with a 27ga luer stub (Instech Laboratories) was gently inserted into the intrathecal space in the midline, dorsal to the spinal cord. The catheter was inserted coinciding with the placement of the distal end of the catheter in proximity to the spinal cord the lumbar vertebrae. The exit end of the catheter is taken out through an opening in the skin and connected to an access port. Rats received 2 mg/kg meloxicam once post surgically and 1 mg/kg daily up to 48 hrs post-surgery if needed. The rats were allowed to recover for 7 days and then motor activity of the rats was examined for any sign of alteration. Competent rats were then randomly assigned to groups and tested with experimental compounds and assessed in behavioral assays. At necropsy after the end of the experiments, catheter placements were ensured by injection of colored dye any nonpatent catheters were excluded from the results.

Chemicals: the peptides were stored at −20° C. in dry powder. The powder was weighed on an analytical balance and an amount of sterile artificial cerebral spinal fluid (acsf, Fischer Scientific) was added to formulate concentrations of 1 mg/mL stock which was diluted to the desired concentration for each individual experiment. Stock solutions were aliquoted and stored at −20° C. until further use. Peptide solutions were delivered with a Hamilton airtight syringe fit with an autoinjector (Instech laboratories) and 10 μL volume of the selected concentration or acsf vehicle was injected intrathecally via the cannula and followed by 100 μL aCSF. The treatments were randomized to include different treatments and controls within the same day experimental setting and observers were blinded to the treatments.

Behavioral assays: on the test day animals were first tested for their baseline score in the open field and then hotplate. The open field assay was conducted in an open-field arena (40W×40L×30H cm) of a 16-square grid clear acrylic open top chamber. Behavior and activity were monitored for 2 min. Activity was assessed by the number of lines each animal crosses with both hind paws and number of rears as a function of time. The purpose of the open field was to ensure there was not a significant change in motor skill due to the cannulation surgery. Open field ambulatory activity was assessed after long hotplate latency in some animals, but it was not quantified as a treatment outcome given the high stimulated state after the nociceptive tests and the difference in duration on the hotplate between treatment and control groups. Thermal nociceptive assay: The thermal nociception was assessed with a hotplate plate with the intensity set at a constant 52.1° C. Animals were placed individually on the warm metal surface and timed until their response of hind paw licking or jumping. A cutoff time limit of 30 s was imposed to prevent tissue damage. After paw licking or jump behavior is observed rats were immediately removed from the hotplate. One trial was used for baseline and timepoint assessment in order to not overstimulate or train the animals to the stimulus. Limiting exposure to the hotplate also ensured that no tissue damage occurred with animals that reached the cutoff.

Chronic pain models: Chemotherapy induced neuropathy was induced in rats with oxaliplatin after i.t. catheter placement recovery with a single i.p. dose of oxaliplatin 6 mg/kg. The animals were allowed to recover for 3 days and then were assessed in the open field assay to ensure motor function and with a von Frey assay to assess allodynia to verify their pain state. The von Frey assay with an electronic aesthesiometer quantified the average baseline for a group of male and female rats to be 72.9±2.7 grams for the mechanical withdrawal threshold after cannulation but before CIPN model induction which fell to 27.9±2.7 grams indicating allodynia. On the day of treatment rats were assessed for baseline measures and then treated and assayed for thermal nociceptive responses.

To study the efficacy of PTx2-3127 (SEQ ID NO: 1) in animal models of pain, the peptide was tested initially in naïve rats to assess the thermal nociceptive responses and monitor open field activity. Doses were selected referencing the in vivo data available for ProTx-II. Merck's study found that ProTx-II had a laming effect via intrathecal administration at 2.4 pg but no effect on nociceptive assays at 0.24 μg i.t. Janssen's study reported that 2 μg in 10 μL ProTx-II was the maximum tolerated dose in rats. Based on this information, a dose of 1.6 μg in 10 μL intrathecal administration to naïve rats was selected. The intrathecal administration was dosed via implanted cannula which were surgically placed in the subarachnoid space of the spinal cord between L4 and L5. After recovery from surgery (−7 days) the rats were assessed for gait and mobility prior to peptide dosing.

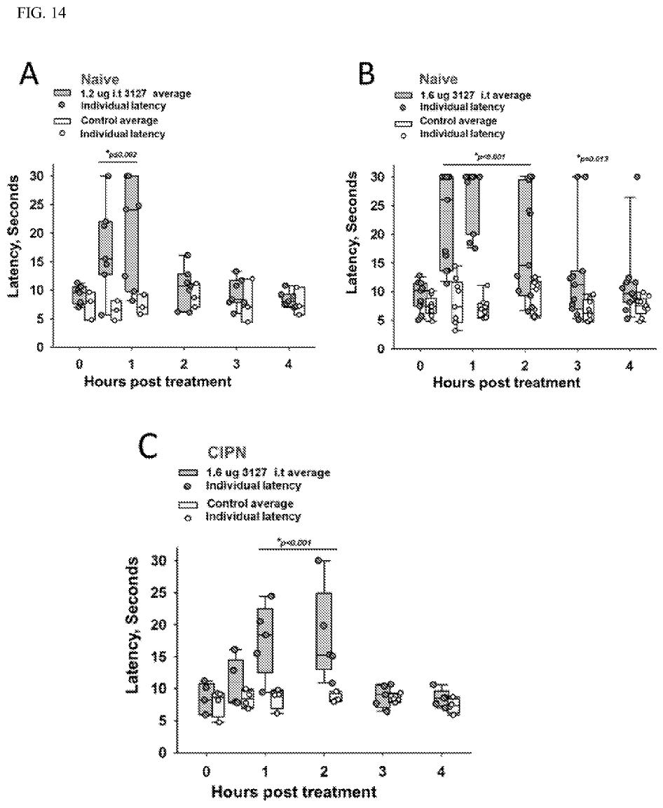

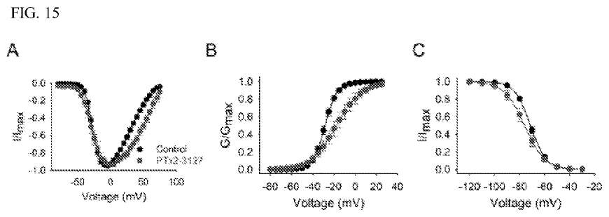

The 1.6 μg dose resulted in robust analgesia with several rats reaching the cutoff latency (30 seconds) for a number of hours on a 52.1° C. hotplate assessed once per hour (FIG. 14) (Two Way Repeated Measures ANOVA, Holm-Sidak method post hoc, p≤0.001 PTx2-3127 (SEQ ID NO: 1) n=11 vs. vehicle n=9). Importantly, this did not lame or significantly alter motor activity of the rats. Rats that timed out per the cutoff were immediately ambulatory after being removed from the hot plate.

The same dose was administered to a group of rats with oxaliplatin induced neuropathy (FIG. 14). These rats with induced chronic pain were assessed on the 52.1° C. hotplate to compare to results from naïve rats. Again the 1.6 μg i.t. dose of PTx2-3127 (SEQ ID NO: 1) resulted in robust analgesia, however with a slightly different time course of action (Two Way Repeated Measures ANOVA, Holm-Sidak method post hoc, p=0.029 3127 n=5 vs. vehicle n=4).

Intrathecal administration of the peptide PTx2-3127 (SEQ ID NO: 1) to otherwise naïve rats blocked pain sensitivity in the suprathreshold hotplate assay over a duration of several hours. After this hotplate assay rats were placed into an open field apparatus where the animals were ambulatory and explorative despite reaching a latency cutoff on the hotplate. The open field was not quantified in this setting because of the supra-stimulation of the hotplate assay directly preceding it. However, these observations correlated with published reports that Nav channel blockade in preclinical models paralleled the human genetic mutant phenotype of pain insensitivity without motor function decrements.

TABLE 7
Sequences.

| SEQ | | |
|---|---|---|
| ID | | |
| NO: | NAME | SEQUENCE |
| 1 | PTx2-3127 | QCQKWMQTCDKDRKCCEGFRCRLWCRKELL |
| 2 | PTx2-3258 | HCQKWMQTCDKDRKCCEGFRCRLWCRKELL |
| 3 | PTx2-3361 | HCQKW-Nle-QTCDKDRKCCEGFRCRLWCRKELL |
| 4 | PTx2-3259 | QCLKWMQTCDKDRKCCEGFRCRLWCRKELL |
| 5 | PTx2-3260 | HCQKWMQTCDKDRKCCEGFRCRLWCR- |
| | | diMePhe-E-tBuCys-L |
| 6 | PTx2-3126 | QCQKAFQTCDKDRKCCEGFRCRLWCRKELL |
| 7 | PTx2-3128 | QCQKWMQTCDKARKCCEGFRCRLWCRKELL |
| 8 | PTx2-2955 | YCQKAFWTCDSERKCCEGLRC-NorR-LWCRKELW |
| 9 | PTx2-3063 | YCQKAFWTCDSARKCCEGLRC-NorR-LWCRKELW |
| 10 | PTx2-3064 | YCQKAFWTCDSARKCCEGLRCRLWCRKELW |
| 11 | PTx2-3065 | YCQKWMQTCDSARKCCEGLRCRLWCRKELW |
| 12 | PTx2-3066 | YCQKWMQTCDKDRKCCEGLRCRLWCRKELL |
| 13 | PTx2-3067 | QCQKWMQTCDSARKCCEGFRCRLWCRKELL |
| 14 | PTx2-2954 | YCQKAFWTCDSERKCCEGLRC-NorR-LWCKKELW |
| 15-19 | blank | |
| 20 | ProTx-II | YCQKWMWTCDSERKCCEGMVCRLWCKKKLW |
| 21 | design1 | HCVLWMQQCDKDRKCCEGLRCRLWCRKELW |
| 22 | design2 | RCVYWMQQCDSTRRCCEGLRCRLWCRKELW |
| 23 | design3 | HCVLWMQQCDSTRRCCEGLRCRLWCRKELW |
| 24 | design4 | HCVLWMQQCDKDRRCCEGLRCRLWCRKELW |
| 25 | design5 | HCVLWMQQCDKDRRCCEGLRCRLWCRKELW |
| 26 | design6 | QCAYWMQQCDKTRRCCEGLRCRLWCRKELW |
| 27 | design7 | QCAYWMQQCDKTRKCCDGLRCRLWCRKELY |
| 28 | design8 | HCVLWMQQCDKDRRCCEGLRCRLWCRKELW |
| 29 | design9 | HCVLWMQQCDKDRRCCEGLRCRLWCRKELW |
| 30 | design10 | HCATWMQQCDKTRKCCDGLRCRLWCRKELW |
| 31 | design11 | HCINWMQQCDSTRRCCEGLRCRLWCRKELW |
| 32 | design12 | QCLYWMQQCDKTRKCCEGLRCRLWCRKELW |
| 33 | design13 | HCVLWMQQCDKDRRCCEGLRCRLWCRKELW |
| 34 | design14 | HCVKWMQQCDKDRRCCEGLRCRLWCRKELW |
| 35 | design15 | HCLLWMQQCDKTRKCCDGLRCRLWCRKELY |
| 36 | design16 | QCAYWMQQCDKTRKCCEGLRCRLWCRKELW |
| 37 | design17 | HCVKWMQQCDSTRKCCEGLRCRLWCRKELW |
| 38 | design18 | RCAYWMQQCDKTRKCCDGLRCRLWCRKELW |
| 39 | design19 | HCQLWMWQCDKDRRCCDGLRCRLWCRKELW |
| 40 | design20 | HCVLWMQQCDKDRRCCEGLRCRLWCRKELW |
| 41 | GsMTx2 | YCQKWMWTCDEERKCCEGLVCRLWCKRIINM |
| 42 | JZTX-V | ECQKWMWTCDSARACCEGLRCKLWCRKII |
| 43 | Heteropodatoxin- | DDCGKLFSGCDTNADCCEGYVCRLWCKLDW |
| | 2 | |
| 44 | ProTx-III | DCLKFGWKCNPRNDKCCSGLKSGSNHNWCKLHL |
| 45 | Huwentoxin-I | ACKGVFDACTPGKNECCPNRVCSDKHKWCKWKL |
| 46 | S67-toxin | GTYCIELGERCPNPREGDWCCHKCVPEGKRFYC |
| | | RDQ |
| 47 | VSTx | ECGKFMWKCKNSNDCCKDLVCSSRWKWCVLASPF |
| 48 | Hainantoxin-I | ECKGFGKSCVPGKNECCSGYACNSRDKWCKVLL |
| 49 | Hainantoxin-III | GCKGFGDSCTPGKNECCPNYACSSKHKWCKVYL |
| 50 | GpTx-1 | DCLGAFRKCIPDNDKCCRPNLVCSRLHRWCKYVF |
| 51 | Jingzhaotoxin-VII | DGECGGFWWKCGRGKPPCCKGYACSKTWGWCAVEAP |
| 52 | Magi-5 | GCKLTFWKCKNKKECCGWNACALGICMPR |
| 53 | GxTx | EGECGGFWWKCGSGKPACCPKYVCSPKWGLCNFPMP |
| 54 | AtracoTx-J | AICTGADRPCAACCPCCPGTSCKAESNGVSYCRKDEP |
| 55 | SGTx | TCRYLFGGCKTTADCCKHLACRSDGKYCAWDGTF |
| 56 | GsMTx4 | GCLEFWWKCNPNDDKCCRPKLKCSKLFKLCNFSF |
| 57 | GrTx | DCVRFWGKCSQTSDCCPHLACKSKWPRNICVWDGSV |
| 58 | Hanatoxin | ECRYLFGGCKTTSDCCKHLGCKFRDKYCAWDFTFS |
| 59 | consensus | GECGGFFWKCDPGKDEDWCCGLVCSSKWKGRKWCKW |
| | | DLPFS |
| 60 | blank | |
| 61 | hNav1.1 753-776 | NTLFMAMEHYPMTDHFNNVLTVGN |
| 62 | hNav1.2 772-795 | NTLFMAMEHYPMTEQFSSVLSVGN |
| 63 | hNav1.3 773-796 | NTLFMAMEHYPMTEQFSSVLTVGN |
| 64 | hNav1.4 591-614 | NTLFMAMEHYPMTEHFDNVLTVGN |
| 65 | hNav1.5 730-753 | NTLFMALEHYNMTXEFEEMLQVGN |
| 66 | hNav1.6 766-789 | NTLFMAMEHHPMTPQFEHVLAVGN |
| 67 | hNav1.7 746-769 | NTLFMAMEHHPMTEEFKNVLAIGN |
| 68 | hNav1.8 678-701 | NTIFMAMEHHGMSPTFEAMLQIGN |
| 69 | hNav1.9 590-613 | NTVFLAMEHHKMEASFEKMLNIGN |
| 70 | consensus | NTLFMAMEHYPMTEQFENVLTVGN |
| 71 | hNav1.1 815-833 | SLVELGLANVEGLSVLRSF |
| 72 | hNav1.2 834-852 | SLMELFLANVEGLSVLRSF |
| 73 | hNav1.3 835-853 | SLMELGLSNVEGLSVLRSF |
| 74 | hNav1.4 653-671 | SLVELGLANVQGLSVLRSF |
| 75 | hNav1.5 792-810 | SLMELGLSRMSNLSVLRSF |
| 76 | hNav1.6 828-846 | SLMELSLADVEGLSVLRSF |
| 77 | hNav1.7 808-826 | SLVELFLADVEGLSVLRSF |
| 78 | hNav1.8 740-758 | SLLELGVAKKGSLSVLRSF |
| 79 | hNav1.9 652-672 | SFADVMNCVLQKRSWPFLRSF |
| 80 | consensus | SLMELGLANLQVEGLSVLRSF |
| 81-100 | blank | |
| 101 | Formula I | X¹-X²-X³-K⁴-X⁵-X⁶-X⁷-X⁸-X⁹-D¹⁰-X¹¹-X¹²-R¹³-K¹⁴-X¹⁵-X¹⁶ |
| | | X¹⁷-G¹⁸-X¹⁹-R²⁰-X²¹-X²²-²³-W²⁴-X²⁵-X²⁶-X²⁷-X²⁸-X²⁹-X³⁰ |
| 102 | Formula II | X¹-C²-X³-K⁴-X⁵-X⁶-X⁷-T⁸-C⁹-D¹⁰-X¹¹-X¹²-R¹³-K¹⁴-C¹⁵-C¹⁶- |
| | | E¹⁷-G¹⁸-X¹⁹-R²⁰-C²¹-X²²-¹²³-W²⁴-C²⁵-X²⁶-X²⁷-E²⁸-X²⁹-X³⁰ |
| 103 | Formula III | X¹-C²-X³-K⁴-X⁵-X⁶-X⁷-T⁸-C⁹-D¹⁰-X¹¹-X¹²-R¹³-K¹⁴-C¹⁵-C¹⁶- |
| | | E¹⁷-G¹⁸-F¹⁹-R²⁰-C²¹-R²²-L-²³-W²⁴-C²⁵-R²⁶-K²⁷-E²⁸-L¹²⁹-L¹³⁰ |
| 104 | Formula IV | X¹-C²-X³-X⁴-W⁵-M⁶-X⁷-Q⁸-C⁹-D¹⁰-X¹¹-X¹²-R¹³-X¹⁴-C¹⁵-C¹⁶- |
| | | X¹⁷-G¹⁸-L¹⁹-R²⁰-C²¹-R²²-L²³-W²⁴-C²⁵-R²⁶-K²⁷-E²⁸-L²⁹-X³⁰ |

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A peptide comprising Formula I
(I)
(SEQ ID NO: 101)
X¹-X²-X³-K⁴-X⁵-X⁶-X⁷-X⁸-X⁹-D¹⁰-X¹¹-X¹²-R¹³-K¹⁴-
X¹⁵-X¹⁶-X¹⁷-G¹⁸-X¹⁹-R²⁰-X²¹-X²²-L-²³-W²⁴-X²⁵-
X²⁶-X²⁷-X²⁸-X²⁹-X³⁰,
or a pharmaceutically acceptable salt thereof,
wherein
X¹ is Q, H, R, K, P, or Y;
X² is C or Sec;
X³ is Q or L;
X⁵ is W or A;
X⁶ is M, Nle, or F;
X⁷ is Q or W;
X⁸ is T or Q;
X⁹ is C or Sec;
X¹¹ is K, R, or S;
X¹² is D, A, T, S, or E;
X¹⁵ is C or Sec;
X¹⁶ is C or Sec;
X¹⁷ is E, D, A, or P;
X¹⁹ is F, norleucine (Nle), or L;
X²¹ is C or Sec;
X²² is R or norarginine (NorR);
X²⁵ is C or Sec;
X²⁶ is R or K;
X²⁷ is K or 2,4-dimethylphenylalanine (diMePhe);
X²⁸ is E or Q;
X²⁹ is L or tert-butylcysteine (tBuCys);
X³⁰ is L, A, C, D, E, F, G, H, I, K, M, N, P, Q, R, S, T, V, W, or Y, or is absent;
when X² and X¹⁶ are each C, the —SH groups between X² and X¹⁶ are combined to form a disulfide bond, or alternatively, X² and X¹⁶ each comprise a —SH group;
when X⁹ and X²¹ are each C, the —SH groups between X⁹ and X²¹ are combined to form a disulfide bond, or alternatively, X⁹ and X²¹ each comprise a —SH group;
when X¹⁵ and X²⁵ are each C, the —SH groups between X¹⁵ and X²⁵ are combined to form a disulfide bond, or alternatively, X¹⁵ and X²⁵ each comprise a —SH group; and
the C-terminus has a —C(O)NH₂, or alternatively, the C-terminus has a —C(O)OH.

2. The peptide of claim 1, or a pharmaceutically acceptable salt thereof, wherein
X⁸ is T.

3. The peptide of claim 1 or 2, or a pharmaceutically acceptable salt thereof, wherein
X¹⁷ is E.

4. The peptide of any one of claims 1 to 3, comprising Formula II:
(II)
(SEQ ID NO: 102)
X¹-C²-X³-K⁴-X⁵-X⁶-X⁷-T⁸-C⁹-D¹⁰-X¹¹-X¹²-R¹³-K¹⁴-
C¹⁵-C¹⁶-E¹⁷-G¹⁸-X¹⁹-R²⁰-C²¹-X²²-L²³-W²⁴-C²⁵-X²⁶-
X²⁷-E²⁸-X²⁹-X³⁰,
or a pharmaceutically acceptable salt thereof,
wherein
X¹ is Q, H, or Y;
X³ is Q or L;
X⁵ is W or A;
X⁶ is M, Nle, or F;
X⁷ is Q or W;
X¹¹ is K or S;
X¹² is D, A, or E;
X¹⁹ is F or L;
X²² is R or norarginine (NorR);
X²⁶ is R or K;
X²⁷ is K or 2,4-dimethylphenylalanine (diMePhe); and
X²⁹ is L or tert-butylcysteine (tBuCys).

5. The peptide of claim 4, or a pharmaceutically acceptable salt thereof, wherein
the —SH groups between C² and C¹⁶ are combined to form a disulfide bond;
the —SH groups between C⁹ and C²¹ are combined to form a disulfide bond; and
the —SH groups between C¹⁵ and C²⁵ are combined to form a disulfide bond.

6. The peptide of any one of claims 1 to 5, or a pharmaceutically acceptable salt thereof, wherein the C-terminus has a —C(O)NH₂.

7. The peptide of any one of claims 1 to 6, or a pharmaceutically acceptable salt thereof, consisting of Formula I or a pharmaceutically acceptable salt thereof.

8. The peptide of any one of claims 1 to 7, or a pharmaceutically acceptable salt thereof, wherein
X¹ is Q or H.

9. The peptide of any one of claims 1 to 8, or a pharmaceutically acceptable salt thereof, wherein
X³ is Q.

10. The peptide of any one of claims 1 to 9, or a pharmaceutically acceptable salt thereof, wherein
X⁵ is W.

11. The peptide of any one of claims 1 to 10, or a pharmaceutically acceptable salt thereof, wherein
X⁶ is M or Nle.

12. The peptide of any one of claims 1 to 11, or a pharmaceutically acceptable salt thereof, wherein
X⁷ is Q.

13. The peptide of any one of claims 1 to 12, or a pharmaceutically acceptable salt thereof, wherein
X¹¹ is K.

14. The peptide of any one of claims 1 to 13, or a pharmaceutically acceptable salt thereof, wherein
X¹² is D.

15. The peptide of any one of claims 1 to 14, or a pharmaceutically acceptable salt thereof, wherein
X¹⁹ is F.

16. The peptide of any one of claims 1 to 15, or a pharmaceutically acceptable salt thereof, wherein
X²² is R.

17. The peptide of any one of claims 1 to 16, or a pharmaceutically acceptable salt thereof, wherein
X²⁶ is R.

18. The peptide of any one of claims 1 to 17, or a pharmaceutically acceptable salt thereof, wherein
X²⁷ is K.

19. The peptide of any one of claims 1 to 18, or a pharmaceutically acceptable salt thereof, wherein
X²⁹ is L.

20. The peptide of any one of claims 1 to 19, or a pharmaceutically acceptable salt thereof, wherein
X³⁰ is L, W, or Y.

21. The peptide of any one of claims 1 to 19, or a pharmaceutically acceptable salt thereof, wherein
X³⁰ is L.

22. The peptide of any one of claims 1 to 21, or a pharmaceutically acceptable salt thereof, comprising Formula III:
(III)
(SEQ ID NO: 103)
X¹-C²-X³-K⁴-X⁵-X⁶-X⁷-T⁸-C⁹-D¹⁰-X¹¹-X¹²-R¹³-K¹⁴-
C¹⁵-C¹⁶-E¹⁷-G¹⁸-F¹⁹-R²⁰-C²¹-R²²-L²³-W²⁴-C²⁵-R²⁶-
K²⁷-E²⁸-L²⁹-L³⁰.

23. The peptide of any one of claims 1 to 22, or a pharmaceutically acceptable salt thereof, wherein the peptide comprises the sequence:
(SEQ ID NO: 1)
QCQKWMQTCDKDRKCCEGFRCRLWCRKELL,
(SEQ ID NO: 2)
HCQKWMQTCDKDRKCCEGFRCRLWCRKELL,
(SEQ ID NO: 3)
HCQKW-Nle-QTCDKDRKCCEGFRCRLWCRKELL,
(SEQ ID NO: 4)
QCLKWMQTCDKDRKCCEGFRCRLWCRKELL,
(SEQ ID NO: 5)
HCQKWMQTCDKDRKCCEGFRCRLWCR-diMePhe-E-tBuCys-L,
(SEQ ID NO: 6)
QCQKAFQTCDKDRKCCEGFRCRLWCRKELL,
(SEQ ID NO: 7)
QCQKWMQTCDKARKCCEGFRCRLWCRKELL,
(SEQ ID NO: 8)
YCQKAFWTCDSERKCCEGLRC-NorR-LWCRKELW,
(SEQ ID NO: 9)
YCQKAFWTCDSARKCCEGLRC-NorR-LWCRKELW,
(SEQ ID NO: 10)
YCQKAFWTCDSARKCCEGLRCRLWCRKELW,
(SEQ ID NO: 11)
YCQKWMQTCDSARKCCEGLRCRLWCRKELW,
(SEQ ID NO: 12)
YCQKWMQTCDKDRKCCEGLRCRLWCRKELL,
(SEQ ID NO: 13)
QCQKWMQTCDSARKCCEGFRCRLWCRKELL,
or
(SEQ ID NO: 14)
YCQKAFWTCDSERKCCEGLRC-NorR-LWCKKELW

24. The peptide of any one of claims 1 to 23, or a pharmaceutically acceptable salt thereof, wherein the peptide comprises the sequence:
(SEQ ID NO: 1)
QCQKWMQTCDKDRKCCEGFRCRLWCRKELL,
(SEQ ID NO: 2)
HCQKWMQTCDKDRKCCEGFRCRLWCRKELL,
or
(SEQ ID NO: 3)
HCQKW-Nle-QTCDKDRKCCEGFRCRLWCRKELL

25. The peptide of any one of claims 1 to 24, or a pharmaceutically acceptable salt thereof, wherein the peptide consists of the sequence:
(SEQ ID NO: 1)
QCQKWMQTCD KDRKCCEGFR CRLWCRKELL

26. The peptide of any one of claims 1 to 24, or a pharmaceutically acceptable salt thereof, wherein the peptide consists of the sequence:
(SEQ ID NO: 2)
HCQKWMQTCDKDRKCCEGFRCRLWCRKELL

27. The peptide of any one of claims 1 to 24, or a pharmaceutically acceptable salt thereof, wherein the peptide consists of the sequence:
(SEQ ID NO: 3)
HCQKW-Nle-QTCDKDRKCCEGFRCRLWCRKELL

28. The peptide of any one of claims 1 to 27, or a pharmaceutically acceptable salt thereof, wherein the peptide inhibits human Nav1.7.

29. The peptide of any one of claims 1 to 28, or a pharmaceutically acceptable salt thereof, wherein the peptide has a human Nav1.7 IC₅₀ of less than about 10000 nM, less than about 1000 nM, less than about 100 nM, or less than about 10 nM in a patch clamp assay.

30. The peptide of any one of claims 1 to 29, or a pharmaceutically acceptable salt thereof, wherein the peptide has a selectivity for human Nav1.7 over human Nav1.1 of at least about 10, at least about 100, at least about 1000, or at least about 10000.

31. The peptide of any one of claims 1 to 30, or a pharmaceutically acceptable salt thereof, wherein the peptide has a selectivity for human Nav1.7 over human Nav1.2 of at least about 10, at least about 100, at least about 1000, or at least about 10000.

32. The peptide of any one of claims 1 to 31, or a pharmaceutically acceptable salt thereof, wherein the peptide has a selectivity for human Nav1.7 over human Nav1.3 of at least about 10, at least about 100, at least about 1000, or at least about 10000.

33. The peptide of any one of claims 1 to 32, or a pharmaceutically acceptable salt thereof, wherein the peptide has a selectivity for human Nav1.7 over human Nav1.4 of at least about 10, at least about 100, at least about 1000, or at least about 10000.

34. The peptide of any one of claims 1 to 33, or a pharmaceutically acceptable salt thereof, wherein the peptide has a selectivity for human Nav1.7 over human Nav1.5 of at least about 10, at least about 100, at least about 1000, or at least about 10000.

35. The peptide of any one of claims 1 to 34, or a pharmaceutically acceptable salt thereof, wherein the peptide has a selectivity for human Nav1.7 over human Nav1.6 of at least about 10, at least about 100, at least about 1000, or at least about 10000.

36. The peptide of any one of claims 1 to 35, or a pharmaceutically acceptable salt thereof, wherein the peptide has a selectivity for human Nav1.7 over human Nav1.8 of at least about 10, at least about 100, at least about 1000, or at least about 10000.

37. The peptide of any one of claims 1 to 36, or a pharmaceutically acceptable salt thereof, wherein the peptide has a selectivity for human Nav1.7 over human Nav1.9 of at least about 10, at least about 100, at least about 1000, or at least about 10000.

38. The peptide of any one of claims 1 to 37, or a pharmaceutically acceptable salt thereof, wherein more than about 50% of the peptide is present after at least about 1 hour, at least about 2 hours, at least about 4 hours, at least about 8 hours, at least about 12 hours, at least about 1 day, at least about 2 days, at least about 3 days, at least about 4 days, or at least about 5 days, in cerebrospinal fluid.

39. A pharmaceutical composition comprising a peptide of any one of claims 1 to 38, or a pharmaceutically acceptable salt thereof, and a pharmaceutically acceptable excipient.

40. A method of treating pain in a subject in need thereof, comprising administering to the subject a therapeutically effective amount of a peptide of any one of claims 1 to 27, or a pharmaceutically acceptable salt thereof.

41. The method of claim 40, wherein the pain is chronic pain.

42. The method of claim 40 or 41, comprising intrathecal, intravenous, or subcutaneous administration of the peptide or pharmaceutically acceptable salt thereof.

43. The method of any one of claims 40 to 42, comprising intrathecal administration of the peptide or pharmaceutically acceptable salt thereof.


SEQUENCE LISTING (104 sequences)

SEQ ID NO: 1 - protein, 30 aa, synthetic construct
QCQKWMQTCDKDRKCCEGFRCRLWCRKELL

SEQ ID NO: 2 - protein, 30 aa, synthetic construct
HCQKWMQTCDKDRKCCEGFRCRLWCRKELL

SEQ ID NO: 3 - protein, 30 aa, synthetic construct
  MOD_RES 6: note = X is norleucine (Nle)
HCQKWXQTCDKDRKCCEGFRCRLWCRKELL

SEQ ID NO: 4 - protein, 30 aa, synthetic construct
QCLKWMQTCDKDRKCCEGFRCRLWCRKELL

SEQ ID NO: 5 - protein, 30 aa, synthetic construct
  MOD_RES 27: note = X is dimethylphenylalanine (diMePhe)
  MOD_RES 29: note = X is tert-butylcysteine (tBuCys)
HCQKWMQTCDKDRKCCEGFRCRLWCRXEXL

SEQ ID NO: 6 - protein, 30 aa, synthetic construct
QCQKAFQTCDKDRKCCEGFRCRLWCRKELL

SEQ ID NO: 7 - protein, 30 aa, synthetic construct
QCQKWMQTCDKARKCCEGFRCRLWCRKELL

SEQ ID NO: 8 - protein, 29 aa, synthetic construct
  MOD_RES 22: note = X is norarginine (NorR)
YCQKAFWTCDSERKCCEGLRCLWCRKELW

SEQ ID NO: 9 - protein, 29 aa, synthetic construct
  MOD_RES 22: note = X is norarginine (NorR)
YCQKAFWTCDSARKCCEGLRCLWCRKELW

SEQ ID NO: 10 - protein, 30 aa, synthetic construct
YCQKAFWTCDSARKCCEGLRCRLWCRKELW

SEQ ID NO: 11 - protein, 30 aa, synthetic construct
YCQKWMQTCDSARKCCEGLRCRLWCRKELW

SEQ ID NO: 12 - protein, 30 aa, synthetic construct
YCQKWMQTCDKDRKCCEGLRCRLWCRKELL

SEQ ID NO: 13 - protein, 30 aa, synthetic construct
QCQKWMQTCDSARKCCEGFRCRLWCRKELL

SEQ ID NO: 14 - protein, 29 aa, synthetic construct
  MOD_RES 22: note = X is norarginine (NorR)
YCQKAFWTCDSERKCCEGLRCLWCKKELW

SEQ ID NO: 15-19 - skipped (5 entries)

SEQ ID NO: 20 - protein, 30 aa, synthetic construct
YCQKWMWTCDSERKCCEGMVCRLWCKKKLW

SEQ ID NO: 21 - protein, 30 aa, synthetic construct
HCVLWMQQCDKDRKCCEGLRCRLWCRKELW

SEQ ID NO: 22 - protein, 30 aa, synthetic construct
RCVYWMQQCDSTRRCCEGLRCRLWCRKELW

SEQ ID NO: 23 - protein, 30 aa, synthetic construct
HCVLWMQQCDSTRRCCEGLRCRLWCRKELW

SEQ ID NO: 24 - protein, 30 aa, synthetic construct
HCVLWMQQCDKDRRCCEGLRCRLWCRKELW

SEQ ID NO: 25 - protein, 30 aa, synthetic construct; identical to SEQ ID NO: 24

SEQ ID NO: 26 - protein, 30 aa, synthetic construct
QCAYWMQQCDKTRRCCEGLRCRLWCRKELW

SEQ ID NO: 27 - protein, 30 aa, synthetic construct
QCAYWMQQCDKTRKCCDGLRCRLWCRKELY

SEQ ID NO: 28 - protein, 30 aa, synthetic construct; identical to SEQ ID NO: 24

SEQ ID NO: 29 - protein, 30 aa, synthetic construct; identical to SEQ ID NO: 24

SEQ ID NO: 30 - protein, 30 aa, synthetic construct
HCATWMQQCDKTRKCCDGLRCRLWCRKELW

SEQ ID NO: 31 - protein, 30 aa, synthetic construct
HCINWMQQCDSTRRCCEGLRCRLWCRKELW

SEQ ID NO: 32 - protein, 30 aa, synthetic construct
QCLYWMQQCDKTRKCCEGLRCRLWCRKELW

SEQ ID NO: 33 - protein, 30 aa, synthetic construct; identical to SEQ ID NO: 24

SEQ ID NO: 34 - protein, 30 aa, synthetic construct
HCVKWMQQCDKDRRCCEGLRCRLWCRKELW

SEQ ID NO: 35 - protein, 30 aa, synthetic construct
HCLLWMQQCDKTRKCCDGLRCRLWCRKELY

SEQ ID NO: 36 - protein, 30 aa, synthetic construct
QCAYWMQQCDKTRKCCEGLRCRLWCRKELW

SEQ ID NO: 37 - protein, 30 aa, synthetic construct
HCVKWMQQCDSTRKCCEGLRCRLWCRKELW

SEQ ID NO: 38 - protein, 30 aa, synthetic construct
RCAYWMQQCDKTRKCCDGLRCRLWCRKELW

SEQ ID NO: 39 - protein, 30 aa, synthetic construct
HCQLWMWQCDKDRRCCDGLRCRLWCRKELW

SEQ ID NO: 40 - protein, 30 aa, synthetic construct; identical to SEQ ID NO: 24

SEQ ID NO: 41 - protein, 31 aa, synthetic construct
YCQKWMWTCDEERKCCEGLVCRLWCKRIINM

SEQ ID NO: 42 - protein, 29 aa, synthetic construct
ECQKWMWTCDSARACCEGLRCKLWCRKII

SEQ ID NO: 43 - protein, 30 aa, synthetic construct
DDCGKLFSGCDTNADCCEGYVCRLWCKLDW

SEQ ID NO: 44 - protein, 33 aa, synthetic construct
DCLKFGWKCNPRNDKCCSGLKSGSNHNWCKLHL

SEQ ID NO: 45 - protein, 33 aa, synthetic construct
ACKGVFDACTPGKNECCPNRVCSDKHKWCKWKL

SEQ ID NO: 46 - protein, 36 aa, synthetic construct
GTYCIELGERCPNPREGDWCCHKCVPEGKRFYCRDQ

SEQ ID NO: 47 - protein, 34 aa, synthetic construct
ECGKFMWKCKNSNDCCKDLVCSSRWKWCVLASPF

SEQ ID NO: 48 - protein, 33 aa, synthetic construct
ECKGFGKSCVPGKNECCSGYACNSRDKWCKVLL

SEQ ID NO: 49 - protein, 33 aa, synthetic construct
GCKGFGDSCTPGKNECCPNYACSSKHKWCKVYL

SEQ ID NO: 50 - protein, 34 aa, synthetic construct
DCLGAFRKCIPDNDKCCRPNLVCSRLHRWCKYVF

SEQ ID NO: 51 - protein, 36 aa, synthetic construct
DGECGGFWWKCGRGKPPCCKGYACSKTWGWCAVEAP

SEQ ID NO: 52 - protein, 29 aa, synthetic construct
GCKLTFWKCKNKKECCGWNACALGICMPR

SEQ ID NO: 53 - protein, 36 aa, synthetic construct
EGECGGFWWKCGSGKPACCPKYVCSPKWGLCNFPMP

SEQ ID NO: 54 - protein, 37 aa, synthetic construct
AICTGADRPCAACCPCCPGTSCKAESNGVSYCRKDEP

SEQ ID NO: 55 - protein, 34 aa, synthetic construct
TCRYLFGGCKTTADCCKHLACRSDGKYCAWDGTF

SEQ ID NO: 56 - protein, 34 aa, synthetic construct
GCLEFWWKCNPNDDKCCRPKLKCSKLFKLCNFSF

SEQ ID NO: 57 - protein, 36 aa, synthetic construct
DCVRFWGKCSQTSDCCPHLACKSKWPRNICVWDGSV

SEQ ID NO: 58 - protein, 35 aa, synthetic construct
ECRYLFGGCKTTSDCCKHLGCKFRDKYCAWDFTFS

SEQ ID NO: 59 - protein, 41 aa, synthetic construct
GECGGFFWKCDPGKDEDWCCGLVCSSKWKGRKWCKWDLPFS

SEQ ID NO: 60 - skipped

SEQ ID NO: 61 - protein, 24 aa, synthetic construct
NTLFMAMEHYPMTDHFNNVLTVGN

SEQ ID NO: 62 - protein, 24 aa, synthetic construct
NTLFMAMEHYPMTEQFSSVLSVGN

SEQ ID NO: 63 - protein, 24 aa, synthetic construct
NTLFMAMEHYPMTEQFSSVLTVGN

SEQ ID NO: 64 - protein, 24 aa, synthetic construct
NTLFMAMEHYPMTEHFDNVLTVGN

SEQ ID NO: 65 - protein, 24 aa, synthetic construct
NTLFMALEHYNMTXEFEEMLQVGN

SEQ ID NO: 66 - protein, 24 aa, synthetic construct
NTLFMAMEHHPMTPQFEHVLAVGN

SEQ ID NO: 67 - protein, 24 aa, synthetic construct
NTLFMAMEHHPMTEEFKNVLAIGN

SEQ ID NO: 68 - protein, 24 aa, synthetic construct
NTIFMAMEHHGMSPTFEAMLQIGN

SEQ ID NO: 69 - protein, 24 aa, synthetic construct
NTVFLAMEHHKMEASFEKMLNIGN

SEQ ID NO: 70 - protein, 24 aa, synthetic construct
NTLFMAMEHYPMTEQFENVLTVGN

SEQ ID NO: 71 - protein, 19 aa, synthetic construct
SLVELGLANVEGLSVLRSF

SEQ ID NO: 72 - protein, 19 aa, synthetic construct
SLMELFLANVEGLSVLRSF

SEQ ID NO: 73 - protein, 19 aa, synthetic construct
SLMELGLSNVEGLSVLRSF

SEQ ID NO: 74 - protein, 19 aa, synthetic construct
SLVELGLANVQGLSVLRSF

SEQ ID NO: 75 - protein, 19 aa, synthetic construct
SLMELGLSRMSNLSVLRSF

SEQ ID NO: 76 - protein, 19 aa, synthetic construct
SLMELSLADVEGLSVLRSF

SEQ ID NO: 77 - protein, 19 aa, synthetic construct
SLVELFLADVEGLSVLRSF

SEQ ID NO: 78 - protein, 19 aa, synthetic construct
SLLELGVAKKGSLSVLRSF

SEQ ID NO: 79 - protein, 21 aa, synthetic construct
SFADVMNCVLQKRSWPFLRSF

SEQ ID NO: 80 - protein, 21 aa, synthetic construct
SLMELGLANLQVEGLSVLRSF

SEQ ID NO: 81-100 - skipped (20 entries)

SEQ ID NO: 101 - protein, 30 aa, synthetic construct
  VARIANT 1: note = Q, H, R, K, P, or Y
  VARIANT 2: note = C or Sec
  VARIANT 3: note = Q or L
  VARIANT 5: note = W or A
  VARIANT 6: note = M, Nle, or F
  VARIANT 7: note = Q or W
  VARIANT 8: note = T or Q
  VARIANT 9: note = C or Sec
  VARIANT 11: note = K, R, or S
  VARIANT 12: note = D, A, T, S, or E
  VARIANT 15: note = C or Sec
  VARIANT 16: note = C or Sec
  VARIANT 17: note = E, D, A, or P
  VARIANT 19: note = F, norleucine (Nle), or L
  VARIANT 21: note = C or Sec
  VARIANT 22: note = R or norarginine (NorR)
  VARIANT 25: note = C or Se
  VARIANT 26: note = R or K
  VARIANT 27: note = K or 2,4-dimethylphenylalanine (diMePhe)
  VARIANT 28: note = E or Q
  VARIANT 29: note = L or tert-butylcysteine (tBuCys)
  VARIANT 30: note = L, A, C, D, E, F, G, H, I, K, M, N, P, Q, R, S, T, V, W, or Y, or is absent
XXXKXXXXXDXXRKXXXGXRXXLWXXXXXX

SEQ ID NO: 102 - protein, 30 aa, synthetic construct
  VARIANT 1: note = Q, H, or Y
  VARIANT 3: note = Q or L
  VARIANT 5: note = W or A
  VARIANT 6: note = M, Nle, or F
  VARIANT 7: note = Q or W
  VARIANT 11: note = K or S
  VARIANT 12: note = D, A, or E
  VARIANT 19: note = F or L
  VARIANT 22: note = R or norarginine (NorR)
  VARIANT 26: note = R or K
  VARIANT 27: note = K or 2,4-dimethylphenylalanine (diMePhe)
  VARIANT 29: note = L or tert-butylcysteine (tBuCys)
XCXKXXXTCDXXRKCCEGXRCXLWCXXEXX

SEQ ID NO: 103 - protein, 30 aa, synthetic construct
  VARIANT 1: note = Q, H, R, K, P, or Y
  VARIANT 3: note = Q or L
  VARIANT 7: note = Q or W
  VARIANT 11: note = K, R, or S
  VARIANT 12: note = D, A, T, S or E
  VARIANT 5: note = W or A
  VARIANT 6: note = M, Nle or F
XCXKXXXTCDXXRKCCEGFRCRLWCRKELL

SEQ ID NO: 104 - protein, 30 aa, synthetic construct
  VARIANT 1: note = Q, H, or R
  VARIANT 3: note = V, A, or L
  VARIANT 4: note = L, Y, K, N, or T
  VARIANT 7: note = Q or W
  VARIANT 11: note = K or S
  VARIANT 12: note = D or T
  VARIANT 14: note = K or R
  VARIANT 17: note = E or D
  VARIANT 30: note = W or Y
XCXXWMXQCDXXRXCCXGLRCRLWCRKELX